(19) United States
(12) Patent Application Publication
(10) Pub. No.: US 2024/0308996 A1
(43) Pub. Date: Sep. 19, 2024

(54) PYRIDOPYRIDINES AND DERIVATIVES THEREOF AS SELECTIVE KINASE INHIBITORS

(71) Applicant: University of Sharjah, Sharjah (AE)

(72) Inventors: Mohammed I. EL-GAMAL, Sharjah (AE); Taleb H. AL-TEL, Sharjah (AE)

(21) Appl. No.: 18/123,009

(22) Filed: Mar. 17, 2023

(51) Int. Cl. C07D 471/04; A61P 35/00

(52) U.S. Cl. CPC C07D 471/04; A61P 35/00

(57) ABSTRACT

Novel pyridopyridine derivative compounds, methods of preparing the compounds, pharmaceutical compositions comprising one or more such compounds, and methods of treatment of diseases associated with protein kinases.

### TECHNICAL FIELD

The present invention relates to novel pyridopyridine derivatives as inhibitors of protein and/or FMS kinase for therapeutic formulations and methods for preventing, treating, or ameliorating diseases such as, cancer, inflammation, arthritis, viral diseases, neurodegenerative diseases such as Alzheimer's disease, cardiovascular diseases, and infectious diseases.

### BACKGROUND OF THE INVENTION

Cancer has become one of the major health challenges that require continuous efforts to develop efficient drugs. It is the second leading cause of death globally after the cardiovascular disorders. The World Health Organization (WHO) expects increment of the number of newly discovered cancer cases to become 15 million cases worldwide every year [2-3].

In the United Arab Emirates, about 4,500 new cancer cases are reported every year. The UAE Government aims at reducing the cancer fatalities to 18% or less by 2021. The most common cancer types in the UAE include leukemia, colorectal, breast, and prostate cancers (https://government.ae/en/information-and-services/health-and-fitness/chronic-diseases-and-natural-disorders/cancer).

Protein kinases are enzymes that serve as key components of signal transduction pathways by catalyzing the transfer of the terminal phosphate from ATP to the hydroxyl group of tyrosine, serine and threonine residues of proteins. Therefore, protein kinase inhibitors and substrates are valuable tools for assessing the physiological consequences of protein kinase activation. The overexpression or inappropriate expression of normal or mutant protein kinases in mammals has been demonstrated to play significant roles in the development of many diseases, including cancer and diabetes. Diabetes, angiogenesis, psoriasis, restenosis, ocular diseases, schizophrenia, rheumatoid arthritis, cardiovascular disease and cancer are exemplary of pathogenic conditions that have been linked with abnormal protein tyrosine kinase activity. Thus, a need exists for selective and potent small-molecule protein tyrosine kinase inhibitors.

### SUMMARY

In accordance with a first aspect of the present invention, there are provided novel compounds that function as inhibitors of FMS kinase for use as anti-cancer agents with antiproliferative activity and high selectivity for cancer cells and as anti-inflammatory agents for the treatment of cardiovascular, inflammatory, and autoimmune diseases.

In a first embodiment of this aspect, there is provided the compound of formula I, or pharmaceutically acceptable salt thereof:

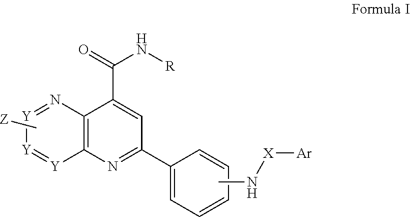

### Where:

R is independently selected from the group consisting of: Me, i-Pr, and 2-pyridil;
X is independently selected from the group consisting of: CO—NH, CS-NH, CO, and SO₂;
Y is independently selected from the group consisting of: CH or CN;
Ar is independently selected from the group consisting of: substituted aromatic, unsubstitued aromatic and heteroaromatic rings;
Z is independently selected from the group consisting of: Alkyl, substituted alkyl, aryl substituted aryl, ether, amin, substituted amine, alogen, and sulfonyl amide.

In a one embodiment of this aspect, there is provided a compound according to formula II, or pharmaceutically acceptable salt thereof:

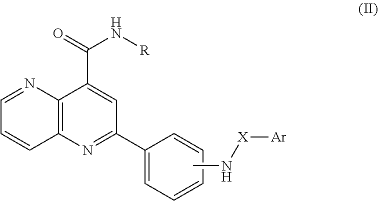

where:
R is independently selected from the group consisting of: Me, i-Pr, and 2-pyridil;
X is independently selected from the group consisting of: CO—NH, CS—NH;
Ar is independently selected from the group consisting of: o-Fluoro(trifluoromethyl)benzene, m-Bis(trifluoromethyl)benzene, 4-[o-(Trifluoromethyl)phenyl]morpholine, 4-Methyl-1-[o-(trifluoromethyl)phenyl]piperazine, and 4-Ethyl-1-[o-(trifluoromethyl)phenyl]piperazine.

In a one embodiment of this aspect, there is provided a compound according to formula 1j, or pharmaceutically acceptable salt thereof:

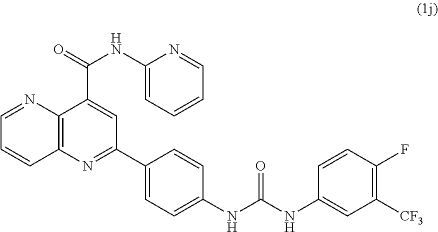 [SMILES: O=C(Nc1ccc(-c2cc(C(=O)Nc3ccccn3)c3ncccc3n2)cc1)Nc1ccc(F)c(C(F)(F)F)c1]

In a one embodiment of this aspect, there is provided a compound according to formula 1jm, or

pharmaceutically acceptable salt thereof:

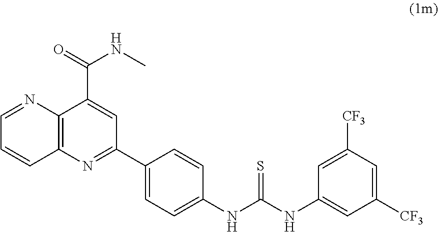 [SMILES: CNC(=O)c1cc(-c2ccc(NC(=S)Nc3cc(C(F)(F)F)cc(C(F)(F)F)c3)cc2)nc2cccnc12]

In a one embodiment of this aspect, there is provided a compound according to formula 13a, or pharmaceutically acceptable salt thereof:

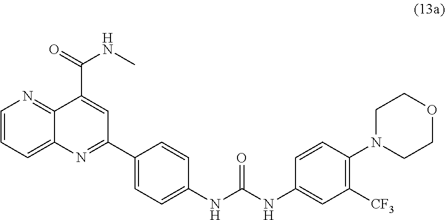 [SMILES: CNC(=O)c1cc(-c2ccc(NC(=O)Nc3ccc(N4CCOCC4)c(C(F)(F)F)c3)cc2)nc2cccnc12]

In a preferred embodiment of this aspect, there is provided a compound according to formula 13b, or pharmaceutically acceptable salt thereof:

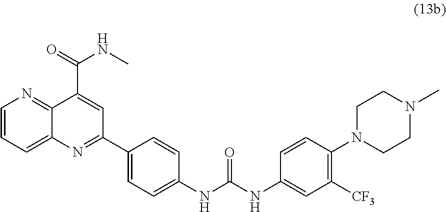 [SMILES: CNC(=O)c1cc(-c2ccc(NC(=O)Nc3ccc(N4CCN(C)CC4)c(C(F)(F)F)c3)cc2)nc2cccnc12]

In a preferred embodiment of this aspect, there is provided a compound according to formula 13c, or pharmaceutically acceptable salt thereof:

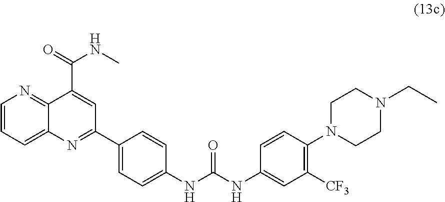 [SMILES: CCN1CCN(c2ccc(NC(=O)Nc3ccc(-c4cc(C(=O)NC)c5ncccc5n4)cc3)cc2C(F)(F)F)CC1]

In a most preferred embodiment of this aspect, there is provided a compound according to formula 13e, or pharmaceutically acceptable salt thereof:

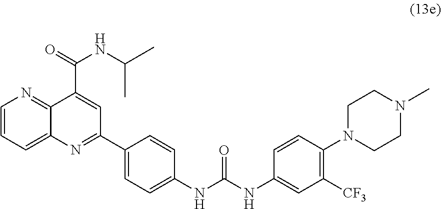 [SMILES: CC(C)NC(=O)c1cc(-c2ccc(NC(=O)Nc3ccc(N4CCN(C)CC4)c(C(F)(F)F)c3)cc2)nc2cccnc12]

In a most preferred embodiment of this aspect, there is provided a compound according to formula 13f, or pharmaceutically acceptable salt thereof:

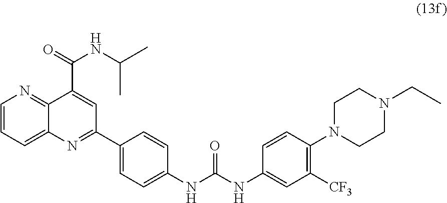 [SMILES: CCN1CCN(c2ccc(NC(=O)Nc3ccc(-c4cc(C(=O)NC(C)C)c5ncccc5n4)cc3)cc2C(F)(F)F)CC1]

In another embodiment of the present disclosure, there is provided a pharmaceutical composition, including a therapeutically effective amount of one or more of the compounds, or pharmaceutically acceptable salts thereof, and one or more pharmaceutical excipients.

In a most preferred embodiment of the present disclosure, there is provided a method of treating a subject afflicted by a disease associated with an altered expression of a kinase, including administering to the subject in need thereof a therapeutically effective amount of any one of compounds 1j, 1m, 13a, 13b, 13 c, 13e, or 13f, a pharmaceutically acceptable salt thereof, and one or more pharmaceutical excipients.

In one aspect of the present disclosure, the compounds of the present disclosure exhibit inhibitory activity against a kinase with an altered expression.

In another aspect of the present disclosure, the inhibitory activity against a kinase with an altered expression is achieved by contacting the kinase with an effective amount of one or more of the compounds of the present disclosure.

In another aspect of the present disclosure, the subject is afflicted by a cancer associated with an abnormal expression of a kinase.

In another aspect of the present disclosure, the subject is afflicted by an inflammatory disease associated with an abnormal expression of a kinase.

In another aspect of the present disclosure, the subject is afflicted by a disease associated with an abnormal expression of a kinase, including cancer, arthritis, Alzheimer, and inflammation, diabetes, psoriasis, angiogenesis, restenosis, and rheumatoid arthritis.

In another aspect of the present disclosure, the subject is afflicted by a metastatic cancer, including ovarian, uterine, breast, prostate, lung, colon, stomach cancer, and hairy cell leukemia.

In a further aspect of the present disclosure, a subject is afflicted by an inflammatory disease associated with an abnormal expression of a kinase selected from the group consisting of protein tyrosine kinase and FMS kinase.

In another aspect of the present disclosure, the subject is a mammal.

In another exemplary aspect of the present disclosure, the mammal is a human.

In a preferred aspect of the present disclosure, a kit for treating a subject with a cancer associated with an altered expression of one or more kinases, including any of the compounds of the present invention or a pharmaceutically acceptable salt thereof and one or more pharmaceutical excipients.

In a preferred embodiment, there is provided a method of synthesizing compounds 1j, 1m, 13a, 13b, 13c, 13e, and 13f, including the following steps:
1. purifying compound 4 from a mixture obtained from Azaisatin 2 reacted with K₂CO₃, compound 3, and KOH;
2. purifying compounds 5a, 5b, and 5c, from a mixture obtained from compound 4, reacted with TEA, TBTU, and R is independently selected from the group consisting of: alkyl, substituted alkyl, aryl, substituted aryl, substituted heteroaromatic rings, unsubstituted heteroaromatic rings.
3. purifying compound 6a, 6b, and 6c, from a mixture obtained from compounds 5a, 5b, and 5c, reacted with NH₄Cl and Fe powder;
4. purifying compounds 1j, 1m, from a mixture of compounds 6a, 6b, 6c, and a solution including isocyanate or isothiocyanate;
5. purifying compounds 8a, 8b, and 8c, from a mixture of compounds 6a, 6b, and phenyl chloroformate;
6. purifying compounds 11a, 11b, and 11c, from a mixture of compounds 9, 10a, 10b, and 10c, an amine including morpholine, N-methylpiperazine, or N-ethylpiperazine, and TEA in THF;
7. purifying compounds 12a, 12b, and 12c, from a mixture obtained from compounds 11a, 11b, and 11c, reacted with Pd/C in THF;
8. purifying compounds 13a, 13b, 13c, 13e, and 13f, from a mixture obtained from compounds 12a, 12b, and 12c, reacted with DIPEA in DMF.

### BRIEF DESCRIPTION OF THE FIGURES

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates the design rationale of the target pyrido[3,2-b]pyridine derivatives.

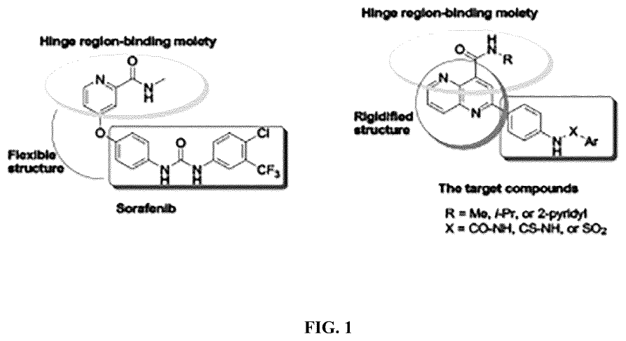

FIG. 2 illustrates reagents and conditions for the synthesis of compounds 1a-q: a) KOH, EtOH, rt, 12 h; b) RNH₂, TBTU, TEA, DMF, −15° C. to rt, 2 h; c) Fe, NH₄Cl, MeOH/H₂O, 80° C., 24 h; d) appropriate aryl isocyanate or isothiocyanate, DMF, rt, overnight; e) appropriate arylsulfonyl chloride, pyridine 0° C. to rt, overnight. The compounds were synthesized and characterized. The synthetic pathways are illustrated in FIG. 1-3, and the structures of the synthesized derivatives are shown in Tables 1-3.

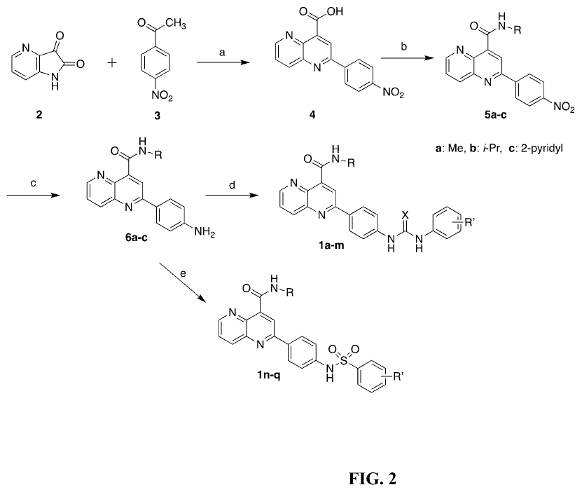

FIG. 3 illustrates reagents and conditions for the synthesis of compounds 13a-f: a) TEA, DCM, 0° C. to rt, 1h; b) TEA, THF, rt, overnight; c) hydrogen gas, Pd/C, rt, 2 h; d) DIPEA, DMF, 40° C., overnight.

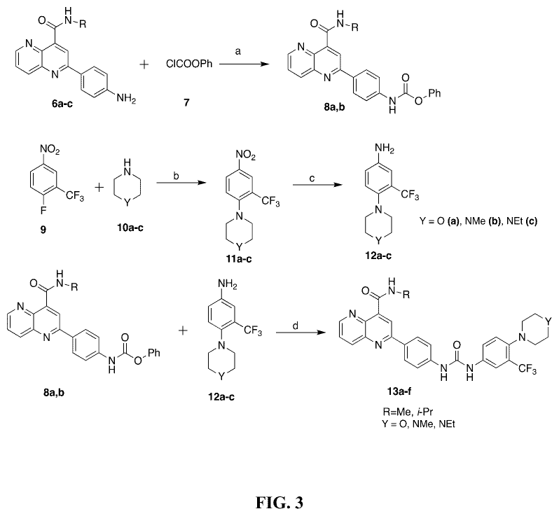

FIG. 4 illustrates reagents and conditions for the synthesis of compounds 13g-13i: a) K2CO₃, DMF, 0° C., 12 h; b) (Boc)₂O, DCM, TEA, rt, 4 h; c) hydrogen gas, Pd/C, THF, rt, 2 h; d) DIPEA, DMF, MS 3 Å, rt, overnight; e) trifluoroacetic acid, DCM, rt, overnight; f) acyloyl chloride, DIPEA, DMF, rt, overnight.

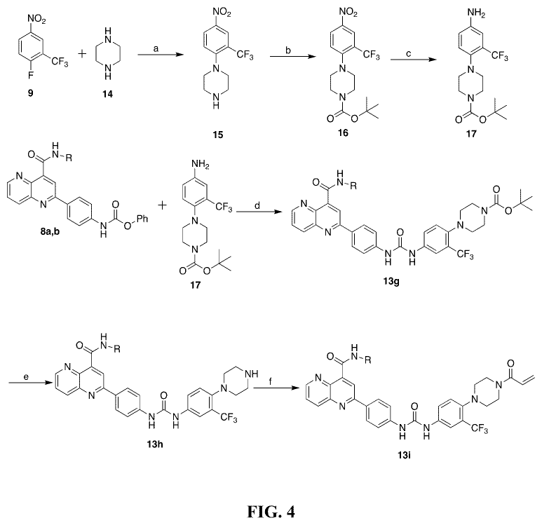

FIG. 5 summarizes the factors for converting doses between animals and human.

### DEFINITIONS

As used in this description and the accompanying claims, the following terms shall have the meanings indicated, unless the context otherwise requires:

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “subject” in accordance with the present invention, includes, e.g., mammals, such as dogs, cats, horses, rats, mice, monkeys, and humans.

The term “treatment” is used conventionally. e.g., the management or care of a subject for the purpose of combating, alleviating, reducing, relieving, improving, etc., one or more of the symptoms associated with a cancer, including all cancers mentioned herein.

The phrase “effective amount” indicates the amount of the compound which is effective to treat any symptom or aspect of the cancer. Effective amounts can be determined routinely. Further guidance on dosages and administration regimens is provided below.

As used herein, the term “therapeutically effective amount” means that amount of a drug or pharmaceutical agent that will elicit the biological or medical response of a tissue, system, animal or human that is being sought, for instance, by a researcher or clinician, and specifically indicates the amount of the compound which is effective to treat any symptom or aspect of cancer, certain precancerous lesions including myelofibrosis, cardiovascular, inflammatory, and autoimmune diseases. Administering effective amounts of the compound can treat one or more aspects of the cancer disease, including, but not limited to, causing tumor regression; causing cell death; causing apoptosis; causing necrosis; inhibiting cell proliferation; inhibiting tumor growth; inhibiting tumor metastasis; inhibiting tumor migration; inhibiting tumor invasion; reducing disease progression; stabilizing the disease; reducing or inhibiting angiogenesis; prolonging patient survival; enhancing patient's quality of life; reducing adverse symptoms associated with cancer; and reducing the frequency, severity, intensity, and/or duration of any of the aforementioned aspects. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Effective amounts can be determined routinely.

### DETAILED DESCRIPTION OF SPECIFIC EMBODIMENTS

In the present disclosure, novel anticancer compounds were synthesized displaying high selectivity against one kinase instead of the promiscuous, multikinase inhibitor sorafenib. The compounds of the present disclosure are novel conformationally-restricted analogues that enhance kinase selectivity.

The compounds of the present disclosure represent novel potent inhibitors of protein tyrosine kinases, such as FMS, and may be useful in the prevention and treatment of disorders resulting from actions of these kinases. The invention also provides methods of making of the compounds of and methods of inhibiting a protein tyrosine kinase comprising contacting the protein tyrosine kinase with an effective inhibitory amount of at least one of the compounds of the present invention. A preferred tyrosine kinase is FMS. In one embodiment of inhibiting a protein tyrosine kinase, at least one of the compounds of the present invention is combined with a known tyrosine kinase inhibitor. The invention further provides methods of treating cancer in mammals, including humans, by administration of a therapeutically effective amount of a pharmaceutically acceptable composition of least one compounds. Exemplary cancers include, but are not limited to, acute myeloid leukemia, acute lymphocytic leukemia, ovarian cancer, uterine cancer, breast cancer, colon cancer, stomach cancer, hairy cell leukemia and non-small lung carcinoma. The invention also provides methods of treating certain precancerous lesions including myelofibrosis. In one embodiment of the invention, an effective amount of at least one compound of the present invention is administered in combination with an effective amount of a chemotherapeutic agent. The invention also provides methods of making the compounds. The invention also provides methods of treating cardiovascular, inflammatory, and autoimmune diseases in mammals, including humans, by administration of a therapeutically effective amount of a pharmaceutically acceptable form of at least one of the compounds of the present invention. Examples of diseases with an inflammatory component include glomerulonephritis, inflammatory bowel disease, prosthesis failure, sarcoidosis, congestive obstructive pulmonary disease, idiopathic pulmonary fibrosis, asthma, pancreatitis, HIV infection, psoriasis, diabetes, tumor related angiogenesis, age-related macular degeneration, diabetic retinopathy, restenosis, schizophrenia or Alzheimer's dementia. These may be effectively treated with compounds of this invention. Other diseases that may be effectively treated include, but are not limited to atherosclerosis and cardiac hypertrophy.

The compounds disclosed herein are rigid. This rigidity leads to reduced conformational rotations, and according to medicinal chemistry rules it leads to increased selectivity. Upon testing some of the target compounds against a panel of 50 kinases, we found that sorafenib as a reference standard exhibited more than 50% inhibition against 22 kinases while the compounds of present invention inhibited three kinases only and the strongest inhibition was reported against FMS kinase. Upon testing the IC₅₀ values, compounds of the formula I showed relative selectivity to FMS kinase. Compounds 13b and 13c are even more potent than the known drug sorafenib against FMS kinase. This enhanced selectivity leads to better safety profile and less side effects. In addition, a few compounds of the present invention demonstrated antiproliferative activity against the tested cancer cell lines.

The compounds of the present disclosure are novel conformationally-restricted analogues with the aim of enhancing kinase selectivity. These compounds exhibit potent inhibiton of protein tyrosine kinases, such as FMS, and may be useful in the prevention and treatment of disorders resulting from actions of these kinases. A preferred tyrosine kinase targeted by the compounds of the present invention is FMS.

In representative embodiments, there is provided a novel class of pyridopyridine derivatives as inhibitors of protein and/or FMS kinase, methods of preparing such compounds, pharmaceutical compositions including one or more such compounds, methods of preparing pharmaceutical formulations including one or more such compounds, and methods of treatment, prevention, inhibition, or amelioration of one or more diseases associated with the protein or FMS kinase using such to treat diseases such as, cancer, inflammation, arthritis, viral diseases, neurodegenerative diseases such as Alzheimer's disease, cardiovascular diseases, and infectious diseases.

The advantage is the ability of the target compounds to kill cancer cells with high selectivity and less toxicity and side effects. So far, no disadvantages appeared.

In a first embodiment of this aspect, there is provided the compound of formula I, or pharmaceutically acceptable salt thereof:

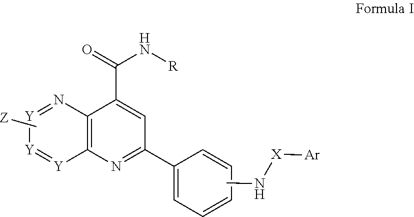

### Where:

R is independently selected from the group consisting of: Me, i-Pr, and 2-pyridil;
X is independently selected from the group consisting of: CO—NH, CS—NH, CO, and SO₂;
Y is independently selected from the group consisting of: CH or CN;
Ar is independently selected from the group consisting of: substituted aromatic, unsubstitued aromatic and heteroaromatic rings;
Z is independently selected from the group consisting of: Alkyl, substituted alkyl, aryl substituted aryl, ether, amin, substituted amine, alogen, and sulfonyl amide.

In a one embodiment of this aspect, there is provided a compound according to formula II, or pharmaceutically acceptable salt thereof:

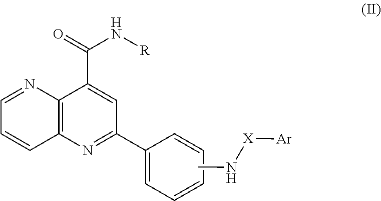

where:
R is independently selected from the group consisting of: Me, i-Pr, and 2-pyridil;
X is independently selected from the group consisting of: CO—NH, CS—NH;
Ar is independently selected from the group consisting of: o-Fluoro(trifluoromethyl)benzene, m-Bis(trifluoromethyl)benzene, 4-[o-(Trifluoromethyl)phenyl]morpholine, 4-Methyl-1-[o-(trifluoromethyl)phenyl]piperazine, and 4-Ethyl-1-[o-(trifluoromethyl)phenyl]piperazine.

In a one embodiment of this aspect, there is provided a compound according to formula 1j, or pharmaceutically acceptable salt thereof:

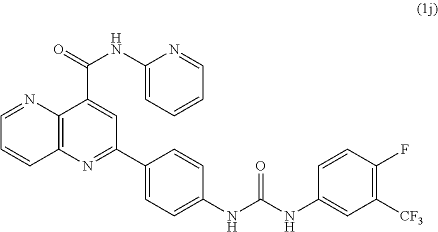 [SMILES: O=C(Nc1ccc(-c2cc(C(=O)Nc3ccccn3)c3ncccc3n2)cc1)Nc1ccc(F)c(C(F)(F)F)c1]

In a one embodiment of this aspect, there is provided a compound according to formula 1jm, or pharmaceutically acceptable salt thereof:

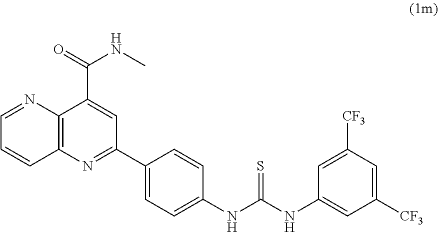 [SMILES: CNC(=O)c1cc(-c2ccc(NC(=S)Nc3cc(C(F)(F)F)cc(C(F)(F)F)c3)cc2)nc2cccnc12]

In a one embodiment of this aspect, there is provided a compound according to formula 13a, or pharmaceutically acceptable salt thereof:

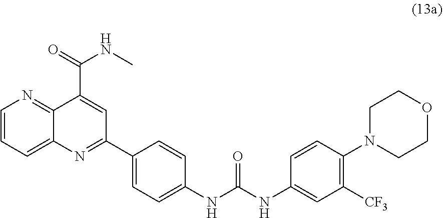 [SMILES: CNC(=O)c1cc(-c2ccc(NC(=O)Nc3ccc(N4CCOCC4)c(C(F)(F)F)c3)cc2)nc2cccnc12]

In a preferred embodiment of this aspect, there is provided a compound according to formula 13b, or pharmaceutically acceptable salt thereof:

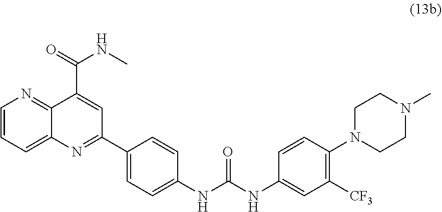 [SMILES: CNC(=O)c1cc(-c2ccc(NC(=O)Nc3ccc(N4CCN(C)CC4)c(C(F)(F)F)c3)cc2)nc2cccnc12]

In a preferred embodiment of this aspect, there is provided a compound according to formula 13c, or pharmaceutically acceptable salt thereof:

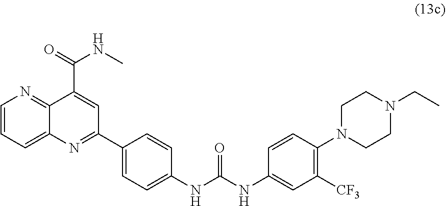 [SMILES: CCN1CCN(c2ccc(NC(=O)Nc3ccc(-c4cc(C(=O)NC)c5ncccc5n4)cc3)cc2C(F)(F)F)CC1]

In a most preferred embodiment of this aspect, there is provided a compound according to formula 13e, or pharmaceutically acceptable salt thereof:

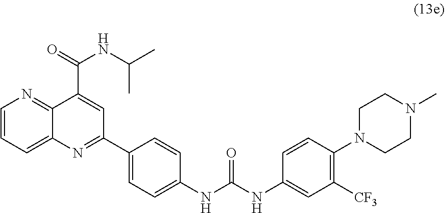 [SMILES: CC(C)NC(=O)c1cc(-c2ccc(NC(=O)Nc3ccc(N4CCN(C)CC4)c(C(F)(F)F)c3)cc2)nc2cccnc12]

In a most preferred embodiment of this aspect, there is provided a compound according to formula 13f, or pharmaceutically acceptable salt thereof:

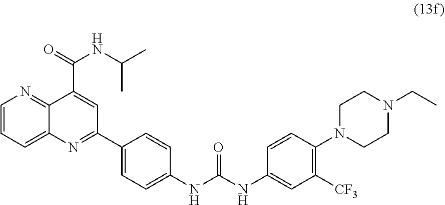 [SMILES: CCN1CCN(c2ccc(NC(=O)Nc3ccc(-c4cc(C(=O)NC(C)C)c5ncccc5n4)cc3)cc2C(F)(F)F)CC1]

In another embodiment of the present disclosure, there is provided a pharmaceutical composition, including a therapeutically effective amount of one or more of the compounds, or pharmaceutically acceptable salts thereof, and one or more pharmaceutical excipients.

In a most preferred embodiment of the present disclosure, there is provided a method of treating a subject afflicted by a disease associated with an altered expression of a kinase, including administering to the subject in need thereof a therapeutically effective amount of any one of compounds 1j, 1m, 13a, 13b, 13 c, 13e, or 13f, a pharmaceutically acceptable salt thereof, and one or more pharmaceutical excipients.

In one aspect of the present disclosure, the compounds of the present disclosure exhibit inhibitory activity against a kinase with an altered expression.

In another aspect of the present disclosure, the inhibitory activity against a kinase with an altered expression is achieved by contacting the kinase with an effective amount of one or more of the compounds of the present disclosure.

In another aspect of the present disclosure, the subject is afflicted by a cancer associated with an abnormal expression of a kinase.

In another aspect of the present disclosure, the subject is afflicted by an inflammatory disease associated with an abnormal expression of a kinase.

In another aspect of the present disclosure, the subject is afflicted by a disease associated with an abnormal expression of a kinase, including cancer, arthritis, Alzheimer, and inflammation, diabetes, psoriasis, angiogenesis, restenosis, and rheumatoid arthritis.

In another aspect of the present disclosure, the subject is afflicted by a metastatic cancer, including ovarian, uterine, breast, prostate, lung, colon, stomach cancer, and hairy cell leukemia.

In a further aspect of the present disclosure, a subject is afflicted by an inflammatory disease associated with an abnormal expression of a kinase selected from the group consisting of protein tyrosine kinase and FMS kinase.

In another aspect of the present disclosure, the subject is a mammal.

In another exemplary aspect of the present disclosure, the mammal is a human.

In a preferred aspect of the present disclosure, a kit for treating a subject with a cancer associated with an altered expression of one or more kinases, including any of the compounds of the present invention or a pharmaceutically acceptable salt thereof and one or more pharmaceutical excipients.

In a most preferred embodiment, there is provided a method of synthesizing compounds 1j, 1m, 13a, 13b, 13c, 13e, and 13f, including the following steps:
1. Adding K₂CO₃ (284.5 mg, 5.07 mmol) dissolved in water (1 mL) to a solution of Azaisatin 2 (500 mg, 3.38 mmol) dissolved in ethanol (10 mL);
2. Stirring the mixture for 30 minutes at room temperature;
3. Detecting complete hydrolysis of Azaisatin by LC-MS;
4. Adding dropwise compound 3 (558.2 mg, 3.38 mmol) dissolved in ethanol (10 mL);
5. Stirring continuously the mixture for 2 hr at room temperature;
6. Adding one equivalent of KOH (189.6 mg, 3.38 mmol) to the mixture;
7. Stirring overnight at room temperature;
8. Detecting completion of the reaction by TLC and LC-MS;
9. Evaporating EtOH in the reaction mixture;
10. Adding dropwise 1M HCl to achieve moderately acidic pH (identified by change in color of pH paper to yellow);
11. Filtering and washing with minimal amount of water;
12. Drying the filtrate under reduced pressure;
13. Purifying compound 4 by normal phase column chromatography, mobile phase: ethyl acetate/hexane 10:90 v/v followed by ethyl acetate/hexane 80:20 v/v;
14. Dissolving compound 4 (150 mg, 0.51 mmol) at −15° C. under N_{2 (g);}
15. Adding TEA (142 μL) dropwise;
16. Stirring the mixture for 15 minutes at room temperature;
17. Adding TBTU (327.5 mg, 1.02 mmol);
18. Stirring the mixture for 30 min and maintaining temperature −15° C. under N_{2 (g).}
19. Adding 5 equivalent of TEA (355 μL) simultaneously to the addition of amine (2.55 mmol);
20. Stirring the mixture for 2 h under N_{2 (g);}
21. Washing the crude compound with ice-cooled H₂O;
22. Filtering the precipitate via vacuum filtration;
23. Drying the filtrate in vacuum desiccator;
24. Purifying compounds 5a-c by column chromatography (silica gel, hexane/ethyl acetate).
25. Adding NH₄Cl (4.9 mmol) and Fe powder (137 mg, 2.45 mmol) to a solution of compound 5a-c (0.49 mmol) in MeOH/H₂O [1:1 v/v] (2.5 mL of each);
26. Stirring for 15 min at room temperature under N_{2 (g);}
27. Heating the mixture at 80° C. overnight;
28. Drying the crude compound under reduced pressure to remove excess metanol;
29. Filtering the compound using ethylacetate;
30. Drying the filtrate in vacuum desiccator;
31. Purifying the filtrate by column chromatography (silica gel, hexane/ethyl acetate);
32. Purifying compound 6a by standard phase column chromatography, mobile phase: ethyl acetate/hexane 10:90 v/v followed by ethyl acetate/hexane 62:38 v/v;
33. Purifying compound 6b by standard phase column chromatography, mobile phase: ethyl acetate/hexane 10:90 v/v followed by ethyl acetate/hexane 55:45 v/v;
34. Purifying compound 6c by standard phase column chromatography, mobile phase: ethyl acetate/hexane 10:90 v/v followed by ethyl acetate/hexane 65:35 v/v;
35. Stirring a mixure of compound 6a-c (0.15 mmol) and isocyanate/isothiocyanate (0.23 mmol) in DMF (2 mL) at 0° C. under N₂ (g) at room temperature overnight.
36. Extracting the reaction mixture by ethyl acetate (3×10 mL) and ice-cooled distilled water (30 mL);
37. Collecting the organic layer;
38. Drying the organic layer over anhydrous Na₂SO₄ and under reduced pressure;
39. Purifying compound 1j by standard phase column chromatography, mobile phase: ethyl acetate/hexane 10:90 v/v followed by ethyl acetate/hexane 65:35v/v;
40. Purifying compound 1m by standard phase column chromatography, mobile phase: ethyl acetate/hexane 10:90 v/v followed by ethyl acetate/hexane 70:30 v/v;
41. Adding TEA (126 mg, 1.25 mmol) at 0° C. to a solution of compound 6a,b (0.5 mmol) in DCM (3.5 mL);
42. Stirring the mixture at 0° C. for 30 min;
43. Adding slowly a solution of phenyl chloroformate (7, 118 mg, 0.75 mmol) in DCM (3.5 mL);
44. Stirring the reaction mixture at room temperature for 1 hr;
45. Extracting the mixture by DCM (3×20 mL) and saturated aqueous NaHCO₃ (35 mL);
46. Collecting the organic layer;
47. Drying the organic layer over anhydrous Na₂SO₄, and under reduced pressure;
48. Stirring a mixture of compound 9 (660 μL, 0.02 mmol) and an amine selected from the group consisting of morpholine, N-methylpiperazine, and N-ethylpiperazine and compound 10a-c (0.07 mmol), and TEA (1.4 mL) in THF (2 mL) at room temperature for 5 min;
49. Stirring the reaction mixture overnight at room temperature;
50. Removing the organic solvent under reduced pressure;
51. Extracting the mixture compounds 11a-c by ethyl acetate (3×10 mL) and ice-cooled distilled water (20 mL);
52. Drying the organic layer over anhydrous Na₂SO₄, and under reduced pressure;
53. Stirring a mixture of compound 11a-c (0.12 mmol) and Pd/C (0.011 mmol, 5%) in THF (2 mL) at room temperature for 2 h under H_{2 (g);}
54. Filtering Pd/C;
55. Evaporating the solvent to obtain a mixture of compounds 12 a-c;
56. Stirring a mixture of 12a-c (0.06 mmol), 4-5 molecular sieves, and DIPEA (230 μL) in DMF (200 μL) at room temperature for 15 min;
57. Adding the mixture of compounds 8a,b (0.08 mmol) while stirring at 40° C. under N_{2 (g)} overnight;
58. Extracting the mixture of compounds 13a-f by ethyl acetate (3×10 mL) and ice-cooled distilled water (20 mL);
59. Collecting the organic layer;
60. Drying the organic layer over anhydrous Na₂SO₄, and under reduced pressure;
61. Purifying compound 13a by standard phase column chromatography, mobile phase: DCM followed by MeOH/DCM 1.8:98.2 v/v;
62. Purifying compound 13b by standard phase column chromatography, mobile phase: DCM 100 v/v followed by MeOH/DCM 1.5:98.5 v/v;
63. Purifying compound 13c by standard phase column chromatography, mobile phase: DCM followed by MeOH/DCM 1.0:99 v/v;
64. Purifying compound 13e by standard phase column chromatography, mobile phase: DCM followed by MeOH/DCM 0.9:99.1 v/v;
65. Purifying compound 13f by standard phase column chromatography, mobile phase: DCM followed by MeOH/DCM 0.6:99.4 v/v.

In a preferred embodiment, there is provided a method of synthesizing compounds 1j, 1m, 13a, 13b, 13c, 13e, and 13f, including the following steps:
1. purifying compound 4 from a mixture obtained from Azaisatin 2 reacted with K₂CO₃, compound 3, and KOH;
2. purifying compounds 5a, 5b, and 5c, from a mixture obtained from compound 4, reacted with TEA, TBTU, and R is independently selected from the group consisting of: alkyl, substituted alkyl, aryl, substituted aryl, substituted heteroaromatic rings, unsubstituted heteroaromatic rings.
3. purifying compound 6a, 6b, and 6c, from a mixture obtained from compounds 5a, 5b, and 5c, reacted with NH₄Cl and Fe powder;
4. purifying compounds 1j, 1m, from a mixture of compounds 6a, 6b, 6c, and a solution including isocyanate or isothiocyanate;
5. purifying compounds 8a, 8b, and 8c, from a mixture of compounds 6a, 6b, and phenyl chloroformate;
6. purifying compounds 11a, 11b, and 11c, from a mixture of compounds 9, 10a, 10b, and 10c, an amine including morpholine, N-methylpiperazine, or N-ethylpiperazine, and TEA in THF;
7. purifying compounds 12a, 12b, and 12c, from a mixture obtained from compounds 11a, 11b, and 11c, reacted with Pd/C in THF;
8. purifying compounds 13a, 13b, 13c, 13e, and 13f, from a mixture obtained from compounds 12 a, 12b, and 12c, reacted with DIPEA in DMF.

In some embodiments, at least one of the compounds of present disclosure can be combined with known tyrosine kinase inhibitors to inhibit a protein tyrosine kinase.

In some embodiments, the compounds of the present disclosure provide methods of treating cancer in mammals, including humans, by administration of a therapeutically effective amount of a pharmaceutically acceptable composition of least one of the compounds presented herein.

Exemplary cancers include, but are not limited to, acute myeloid leukemia, acute lymphocytic leukemia, ovarian cancer, uterine cancer, breast cancer, colon cancer, stomach cancer, hairy cell leukemia and non-small lung carcinoma. The invention also provides methods of treating certain precancerous lesions including myelofibrosis. In one embodiment of the invention, an effective amount of at least one compound of the present invention is administered in combination with an effective amount of a chemotherapeutic agent. The invention also provides methods of making the compounds. The invention also provides methods of treating cardiovascular, inflammatory, and autoimmune diseases in mammals, including humans, by administration of a therapeutically effective amount of a pharmaceutically acceptable form of at least one of the compounds of the present invention. Examples of diseases with an inflammatory component include glomerulonephritis, inflammatory bowel disease, prosthesis failure, sarcoidosis, congestive obstructive pulmonary disease, idiopathic pulmonary fibrosis, asthma, pancreatitis, HIV infection, psoriasis, diabetes, tumor related angiogenesis, age-related macular degeneration, diabetic retinopathy, restenosis, schizophrenia or Alzheimer's dementia. These may be effectively treated with compounds of this invention. Other diseases that may be effectively treated include but are not limited to atherosclerosis and cardiac hypertrophy.

In one embodiment, disclosed is herein the structure of compounds 1a-m (Table 1):

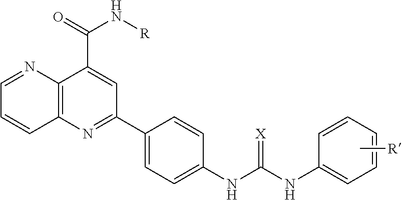

TABLE 1

| Compound | | | |
|---|---|---|---|
| No. | R | X | R′ |
| 1a | Me | O | 4-F |
| 1b | Me | O | 3,4-Cl₂ |
| 1c | Me | O | 3-CF₃, 4-F |
| 1d | Me | O | 3-CF₃, 4-Cl |
| 1e | Me | O | 3,5-(CF₃)₂ |
| 1f | i-Pr | O | 4-F |
| 1g | i-Pr | O | 3-CF₃, 4-F |
| 1h | i-Pr | O | 3,5-(CF₃)₂ |
| 1i | 2-pyridyl | O | 4-F |
| 1j | 2-pyridyl | O | 3-CF₃, 4-F |
| 1k | 2-pyridyl | O | 3,5-(CF₃)₂ |
| 1l | Me | S | 3-CF₃, 4-Cl |
| 1m | Me | S | 3,5-(CF₃)₂ |

In one embodiment, disclosed is herein the structure of compounds 1n-q (Table 2):

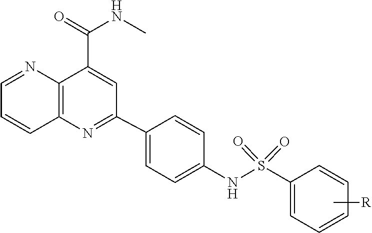

TABLE 2

| Compound | |
|---|---|
| No. | R |
| 1n | 4-F |
| 1o | 4-Me |
| 1p | 4-tert-butyl |
| 1q | 3,5-(CF₃)₂ |

In one embodiment, disclosed is herein the structure of compounds 13a-f (Table 3):

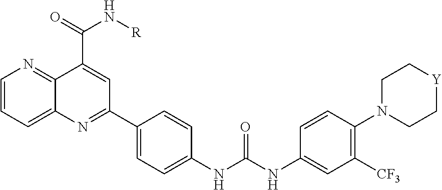

TABLE 3

| Compound | | |
|---|---|---|
| No. | R | Y |
| 13a | Me | O |
| 13b | Me | N-Me |
| 13c | Me | N-Et |
| 13d | i-Pr | O |
| 13e | i-Pr | N-Me |
| 13f | i-Pr | N-Et |

Compositions featuring the above mentioned compounds may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin. propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations may conveniently be presented in unit dosage form and may be prepared by any methods well known in the art of pharmacy. The amount of compound which can be combined with a carrier material to produce a single dosage form will vary depending upon the subject being treated, the particular mode of administration. The amount of an active ingredient which can be combined with a carrier material to produce a single dosage form will usually be that amount of the compound which produces a therapeutic effect. Usually, out of one hundred per cent, this amount will range from about 1 wt % to about 99 wt % of active ingredient, preferably from about 5 wt % to about 70 wt %, most preferably from about 10 wt % to about 30 wt %.

In certain embodiments, a formulation of the compound includes an excipient selected from the group consisting of cyclodextrins, liposomes, micelle forming agents, e.g., bile acids, and polymeric carriers, e.g., polyesters and polyanhydrides; and an active ingredient that may be the compound and/or one of its pharmaceutically acceptable derivatives. In certain embodiments, an aforementioned formulation renders orally bioavailable a compound or its derivative.

Methods of preparing these formulations or compositions include the step of bringing into association the compound with the carrier and, optionally, one or more accessory ingredients. Usually, the formulations are prepared by uniformly and intimately bringing into association a compound of the present invention with liquid carriers, or finely divided solid carriers, or both, and then, if necessary, shaping the product.

Liquid dosage forms for oral administration of the compound include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A formulation of the compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also include buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

The tablets, and other solid dosage forms of the formulation of the compound, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Formulations of the pharmaceutical compositions of the compound for rectal or vaginal administration may be presented as a suppository, which may be prepared by the compound with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for the topical or transdermal administration of the compound include powders, sprays, ointments, pastes, creams, lotions, gels, solutions, patches and inhalants. The extract may be mixed under sterile conditions with a pharmaceutically-acceptable carrier, and with any preservatives, buffers, or propellants which may be required. The ointments, pastes, creams and gels may contain, in addition to an extract, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an extract, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the compound to the body. Such dosage forms can be made by dissolving or dispersing an extract in the proper medium. Absorption enhancers can also be used to increase the flux of the extract or dispersing the extract in a polymer matrix or gel.

Pharmaceutical compositions suitable for parenteral administration include one or more components of the compound in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the compound may be formulated into pharmaceutically-acceptable dosage forms by conventional methods known to those of skill in the art. The compound may be formulated for administration in any convenient way for use in human or veterinary medicine, by analogy with other pharmaceuticals.

In certain embodiments, the above-described pharmaceutical compositions include the compound, a chemotherapeutic agent, and optionally a pharmaceutically acceptable carrier. Alternatively, the terms “chemotherapeutic agent” or “therapeutic agent” include, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

### Methods of Cancer Treatment

The above described compositions may be used in novel therapeutic methods of treatment in cancer patients. The methods include administering to a subject an effective amount of a pharmaceutical compound composition. In representative embodiments, the subject suffers from a liver cancer. In specific embodiments, the type of liver cancer is a hepatocellular carcinoma (HCC), fibrolamellar HCC, cholangiocarcinoma, angiosarcoma, or liver cancer.

The above invention can be used to treat any cancer irrespective of the type or cause of the cancer, and irrespective of the genetic lesions associated with it, including, but not limited to cancer, pre-cancerous cells, tumors, neoplasms, and non-malignant tumors can also be treated. Cancers that can be treated include, e.g., cancers which are primary; which arise from a primary tumor at a secondary metastatic site; which have been treated by surgery (e.g., entirely removed, surgical resection, etc); which have been treated by chemotherapy, radiation, radiofrequency ablation, and/or any other adjunct to drug therapy; which have acquired drug-resistance; which are refractory to a chemotherapeutic agent.

In other embodiments, types of cancer which can be treated in accordance with present invention include, but are not limited to: Cell Adult Acute Lymphoblastic Leukemia; Blastic Phase Chronic Myelogenous Leukemia: Bone Metastases; Brain Tumor; Breast Cancer; Cancer; Central Nervous System Cancer; Childhood Acute Lymphoblastic Leukemia; Childhood Acute Lymphoblastic Leukemia in Remission; Childhood Central Nervous System Germ Cell Tumor; Childhood Chronic Myelogenous Leukemia; Childhood Soft Tissue Sarcoma; Chordoma; Chronic Eosinophilic Leukemia (CEL): Chronic Idiopathic Myelofibrosis; Chronic Myelogenous Leukemia; Chronic Myeloid Leukemia; Chronic Myelomonocytic Leukemia; Chronic Phase Chronic Myelogenous Leukemia; Colon Cancer; Colorectal Cancer; Dermatofibrosarcoma; Dermatofibrosarcoma Protuberans (DFSP): Desmoid Tumor; Eosinophilia: Epidemic Kaposi's Sarcoma; Essential Thrombocythemia: Ewing's Family of Tumors; Extensive Stage Small Cell Lung Cancer; Fallopian Tube Cancer; Familiar Hypereosinophilia; Fibrosarcoma; Gastric Adenocarcinoma; Gastrointestinal Neoplasm; Gastrointestinal Stromal Tumor; Glioblastoma; Glioma; Gliosarcoma; Grade I Meningioma; Grade Il Meningioma; Grade III Meningioma; Hematopoietic and Lymphoid Cancer, High-Grade Childhood Cerebral Astrocytoma; Hypereosinophilic Syndrome; Idiopathic Pulmonary Fibrosis; L1 Adult Acute Lymphoblastic Leukemia; L2 Adult Acute Lymphoblastic Leukemia; Leukemia, Lymphocytic, Acute L2; Leukemia, Myeloid, Chronic; Leukemia, Myeloid, Chronic Phase; Liver Dysfunction and Neoplasm; Lung Disease; Lymphoid Blastic Phase of Chronic Myeloid Leukemia; Male Breast Cancer; Malignant Fibrous Histiocytoma; Mastocytosis; Meningeal Hemangiopericytoma; Meningioma; Meningioma; Meningioma; Metastatic Cancer; Metastatic Solid Tumors; Myelofibrosis; Myeloid Leukemia, Chronic; Myeloid Leukemia, Chronic Accelerated-Phase; Myeloid Leukemia, Chronic, Chronic-Phase; Myeloid Metaplasia; Myeloproliferative Disorder (MPD) with Eosinophilia; Neuroblastoma; Noπ-T, Non-B Childhood Acute Lymphoblastic Leukemia; Oligodendroglioma; Osteosarcoma; Ovarian Germ Cell Tumor; Ovarian Low Malignant Potential Tumor; Ovarian Neoplasms; Pancreatic Cancer; Pelvic Neoplasms; Peritoneal Cavity Cancer; Peritoneal Neoplasms; Philadelphia Chromosome Positive Chronic Myelogenous Leukemia; Philadelphia Positive Acute Lymphoblastic Leukemia; Philadelphia Positive Chronic Myeloid Leukemia in Myeloid Blast Crisis; Polycythemia Vera; Pulmonary Fibrosis; Recurrent Adult Brain Tumor; Recurrent Adult Soft Tissue Sarcoma; Recurrent Breast Cancer; Recurrent Colon Cancer; Recurrent Esophageal Cancer; Recurrent Gastric Cancer; Recurrent Glioblastoma Multiforme (GBM); Recurrent Kaposi's Sarcoma; Recurrent Melanoma; Recurrent Merkel Cell Carcinoma; Recurrent Ovarian Epithelial Cancer; . Recurrent Pancreatic Cancer; Recurrent Prostate Cancer; Recurrent Rectal Cancer; Recurrent Salivary Gland Cancer; Recurrent Small Cell Lung Cancer; Recurrent Tumors of the Ewing's Family; Recurrent Uterine Sarcoma; Relapsing Chronic Myelogenous Leukemia; Rheumatoid Arthritis; Salivary Gland Adenoid Cystic Carcinoma; Sarcoma; Small Cell Lung Cancer; Stage U Melanoma; Stage Ii Merkel Cell Carcinoma; Stage III Adult Soft Tissue Sarcoma; Stage III Esophageal Cancer; Stage 111 Merkel Cell Carcinoma; Stage ill Ovarian Epithelial Cancer; Stage III Pancreatic Cancer; Stage III Salivary Gland Cancer; Stage IUB Breast Cancer; Stage UIC Breast Cancer; Stage IV Adult Soft Tissue Sarcoma; Stage IV Breast Cancer; Stage IV Colon Cancer; Stage IV Esophageal Cancer; Stage IV Gastric Cancer; Stage IV Melanoma; Stage IV Ovarian Epithelial Cancer; Stage IV Prostate Cancer; Stage IV Rectal Cancer; Stage IV Salivary Gland Cancer; Stage IVA Pancreatic Cancer; Stage IVB Pancreatic Cancer; Systemic Mastocytosis; T-Cell Childhood Acute Lymphoblastic Leukemia; Testicular Cancer; Thyroid Cancer; Unresectable or Metastatic Malignant

Gastrointestinal Stromal Tumor (GIST); Unspecified Adult Solid Tumor; Untreated Childhood Brain

Stem Glioma; Uterine Carcinosarcoma, and Uterine Sarcoma. Diseases which can be treated in accordance with present invention include, e.g., diseases which are treated with gefitinib. such as, but not limited to; Adenocarcinoma of the Colon; Adenocarcinoma of the Esophagus; Adenocarcinoma of the Lung; Adenocarcinoma of the Prostate; Adenocarcinoma of the Rectum; Advanced Adult Primary Liver Cancer; Advanced Non-Nasopharyngeal Head and Neck Carcinoma; Anaplastic Astrocytoma; Anaplastic Oligodendroglima; Anaplastic Thyroid Cancer; Bladder Cancer; Brain Tumor; Breast Cancer; Breast Cancer in Situ; Breast Neoplasms; Bronchoalveolar Cell Lung Cancer; Cancer of the Fallopian Tube; Carcinoma. Squamous Cell; Cervix Neoplasms; Colon Cancer; Colorectal Cancer; Epithelial Mesothelioma; Esophageal Cancer; Esophagogastric Cancer; Follicular Thyroid Cancer; Gastric Cancer; Gastrinoma; Gastrointestinal Carcinoid; Giant Cell Glioblastoma; Glioblastoma; Glioblastoma Multiforme; Head and Neck Cancer; Hepatocellular Carcinoma; Hypopharyngeal Cancer; Inoperable Locally Advanced Squamous Cell Carcinoma of Head and Neck; Insulinoma; Intraductal Breast Carcinoma; Islet Cell Carcinoma; Large Cell Lung Cancer; Laryngeal Cancer; Lip and Oral Cavity Cancer; Lip Cancer; Liver Cancer; Lung Adenocarcinoma With Bronchiole-Alveolar Feature; Lung Cancer; Male Breast Cancer; Medullary Thyrod Cancer; Meningeal Tumors; Metastatic Colorectal Cancer; Metastatic Gastrointestinal Carcinoid Tumor; Metastatic Pancreatic Carcinoma; Mixed Gliomas; Myelogenous Leukemia. Acute; Nasopharyngeal Carcinoma; Neuroblastoma; Non-Metastatic (T2-T4, N0-N3. MO; Stages Il and III) and Histologically-Confirmed Intestinal GC; Non-Metastatic Prostate Cancer; Nonresectable Adrenocortical Carcinoma; Non-Small Cell Lung Cancer; Nose Cancer; Oligodendroglial Tumors; Oral Cancer; Oropharyngeal Cancer; Osteosarcoma; Ovarian Cancer; Ovarian Neoplasms; Pancreatic Cancer; Papillary Thyroid Cancer; Peritoneal Carcinoma; Pharynx Cancer; Pneumonic-Type Adenocarcinoma (P-ADC); Primary Hepatocellular Carcinoma; Prostate Cancer; Rectal Cancer; Recurrent Adult Primary Liver Cancer; Recurrent Breast Cancer; Recurrent Colon Cancer; Recurrent Endometrial Cancer, Recurrent Esophageal Cancer; Recurrent Glioblastoma; Recurrent Rectal Cancer; Recurrent Skin Cancer; Refractory Germ Cell Tumors Expressing EGRF; Renal Cell Cancer; Rhabdomyosarcomas; Sarcomatous Mesothelioma; Skin Cancer; Soft Tissue Sarcoma; Squamous Cell Carcinoma of the Esophagus; Squamous Cell Carcinoma of the Head and Neck; Squamous Cell Carcinoma of the Skin; Squamous Cell Lung Cancer; Stage II Esophageal Cancer; Stage III Esophageal Cancer, Synovial Sarcoma; Thorax and Respiratory Cancer; Throat Cancer; Thyroid Cancer; Transitional Cell Cancer of the Renal Pelvis and Ureter; Transitional Cell Carcinoma of the Bladder; Tubal Carcinoma; Unspecified Childhood Solid Tumor; Untreated Childhood Brain Stem Glioma; Urethral Cancer.

As anticipated above, the compound may be administered by any appropriate route, for example orally, parenterally, topically, or rectally. It will be appreciated that the preferred route may vary with, for example, the condition of the recipient of the compound and the cancer to be treated. In certain embodiments, the extract may be especially suitable for the preparation of pharmaceuticals for intravenous administration, such as intravenous injection or infusion, provided that it does not contain components with serum-precipitating and/or haemagglutinating properties which disturb such an application. The extract may therefore be provided in the form of ampoule preparations which are directed to intravenous administration. In still other embodiments, the method comprises systemic administration of a subject composition to a subject.

Also provided are methods of treating cancer, for example liver cancer, which include administering any one of the compounds of the present invention in conjunction with a chemotherapeutic agent to a subject. Conjunctive therapy includes sequential, simultaneous and separate, or co-administration of the compounds and the chemotherapeutic agent in a way that the therapeutic effect of the chemotherapeutic agent is not entirely disappeared when the compound is administered. In certain embodiments, the compounds and the chemotherapeutic agent may be compounded together in the same unitary pharmaceutical composition including both entities. Alternatively, the combination of any one of the compounds and chemotherapeutic agent may be administered separately in separate pharmaceutical compositions, each including one of the compound and chemotherapeutic agent in a sequential manner wherein, for example, the compound or the chemotherapeutic agent is administered first and the other second.

Exemplary doses of the compound in the range from about 0.001, 0.01, 0.1, 0.5, 1, 10, 15, 20, 25, 50, 100, 200, 300, 400, 500, 600, or 750 to about 1000 mg/day per kg body weight of the subject. In certain embodiments, the dose of the compound will typically be in the range of about 100 mg/day to about 1000 mg/day per kg body weight of the subject, specifically in the range of about 200 mg/day to about 750 mg/day per kg, and more specifically in the range of about 250 mg/day to about 500 mg/day per kg. In an embodiment, the dose is in the range of about 50 mg/day to about 250 mg/day per kg. In a further embodiment, the dose in the range of about 100 mg/day to about 200 mg/day per kg. In an embodiment, the dose is in the range of about 15 mg/day to 60 mg/day per kg. In a further embodiment, the dose is in the range of about 20 mg/day to 50 mg/day per kg. In an additional embodiment, the dose is in the range of about 25 mg/day to 45 mg/day per kg.

The combined use of the compound and other chemotherapeutic agents may reduce the required dosage for any individual component because the onset and duration of effect of the different components may be complementary. In such combination therapies, the different active agents may be delivered together or separately, and simultaneously or at different times within the day.

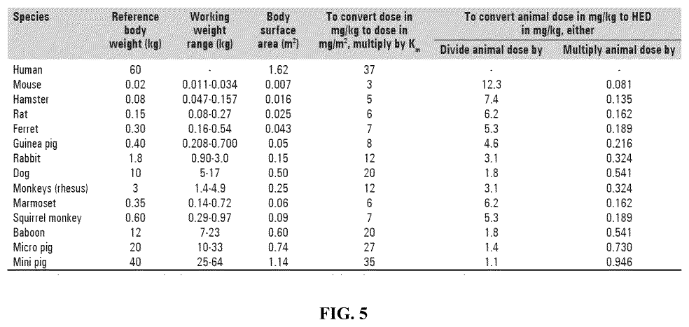

The data obtained from cell culture assays and animal studies may be used in formulating a range of dosage for use in humans. For example, effective dosages achieved in one animal species may be extrapolated for use in another animal, including humans, as illustrated in the conversion table of FIG. 5 where human equivalent dose (HED) dosage factors based on body surface area of other species are reported [3]. The dosage of any supplement, or alternatively of any components therein, lies preferably within a range of circulating concentrations that include the ED₅₀ with little or no toxicity. The dosage may vary within this range depending upon the dosage form employed and the route of administration utilized. For the compound or combinations of the compound and other chemotherapeutic agents, the therapeutically effective dose may be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

In a third aspect of the present disclosure, there is provided a kit for treating a subject afflicted by a cancer associated with an altered expression of one or more kinases, the kit comprising the any one of the compounds of the present invention, or a pharmaceutically acceptable salt thereof, and one or more pharmaceutical excipients.

The present invention provides kits for novel therapeutic methods in cancer patients. For example, a kit may include one or more pharmaceutical compositions of the compound as described above. The compositions may be pharmaceutical compositions comprising a pharmaceutically acceptable excipient. In other embodiments involving kits, this invention provides a kit including the compound, optionally a chemotherapeutic agent, and optionally instructions for their use in the treatment of cancer. In still other embodiments, the invention provides a kit comprising one more pharmaceutical compositions and one or more devices for accomplishing administration of such compositions. For example, a subject kit may comprise a pharmaceutical composition and catheter for accomplishing direct intraarterial injection of the composition into a cancer. In an embodiment, the device is an intraarterial catheter. Such kits may have a variety of uses, including, for example, therapy, diagnosis, and other applications.

### EXPERIMENTAL EXAMPLES

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

Compounds 13b, 13c, 13e, 13f, were tested against NCI-60 cell lines for in-vitro anti-cancer activity (Tables 3-6). Table 4 shows inhibition percentage values of compound 13b.

Table 5 shows inhibition percentage values of compound 13c.

Table 6 shows inhibition percentage values of compound 13e.

Table 7 shows inhibition percentage values of compound 13f.

Compounds 13b. 13c. 13e. 13f. and sorafenib were tested over the NCI cancer cell line panel for half maximal inhibition concentration (IC50) and for total growth inhibition (TGI) (Table 8).

TABLE 8

| | | Comp. 13b | | Comp. 13c | | Comp. 13e | | Comp. 13f | | Sorafenib | |
|---|---|---|---|---|---|---|---|---|---|---|---|
| Cancer Type | Cell Line | IC₅₀^{a} | TGI^{b} | IC₅₀^{a} | TGI^{b} | IC₅₀^{a} | TGI^{b} | IC₅₀^{a} | TGI^{b} | IC₅₀^{a} | TGI^{b} |
| Leukemia | CCRF-CEM | 3.16 | 74.1 | 0.46 | 1.27 | 0.74 | 1.98 | 1.01 | 3.10 | 2.00 | 5.01 |
| | HL-60(TB) | 16.2 | 38.7 | 3.42 | >18.0 | 0.77 | 2.12 | 1.07 | 5.89 | 1.58 | 100 |
| | K-562 | 2.12 | 5.35 | 0.66 | >18.0 | 0.92 | >38.8 | 1.00 | >34.5 | 3.16 | NT^{c} |
| | MOLT-4 | 2.79 | 14.9 | 0.70 | >18.0 | 0.76 | 2.89 | 1.11 | 30.60 | 3.16 | 100 |
| | RPMI-8226 | 3.85 | 25.0 | 3.05 | >18.0 | 0.98 | 3.48 | 1.04 | >34.5 | 1.58 | 3.16 |
| | SR | 9.97 | 44.6 | 0.67 | >18.0 | 1.00 | 3.19 | 1.05 | 12.10 | 3.16 | 63.10 |
| Non-Small | A549/ATCC | 10.9 | 24.3 | 0.47 | 1.30 | 0.73 | 1.64 | 1.14 | 5.77 | 3.16 | 7.94 |
| Cell Lung | EKVX | 17.9 | 33.9 | 2.94 | 5.63 | 0.77 | 1.49 | 1.61 | 5.82 | 2.51 | 19.95 |
| Cancer | HOP-62 | 16.4 | 32.2 | 2.79 | 5.41 | 0.87 | 1.94 | 3.83 | 8.89 | 2.00 | 3.16 |
| | HOP-92 | 6.54 | 100 | 2.45 | 5.67 | 0.70 | 1.33 | 3.14 | 8.29 | 1.58 | 5.01 |
| | NCI-H226 | 18.0 | 39.7 | 2.70 | 6.36 | 5.60 | 14.7 | 5.02 | 12.50 | 2.00 | 3.16 |
| | NCI-H23 | NT^{c} | NT^{c} | 2.68 | 5.68 | 0.73 | 1.46 | 2.05 | 7.27 | 2.00 | 5.01 |
| | NCI-H322M | 15.6 | 29.8 | 2.80 | 5.20 | 0.71 | 1.55 | 1.85 | 7.24 | 2.51 | 5.01 |
| | NCI-H460 | 3.19 | 10.2 | 0.66 | 2.93 | 0.70 | 1.31 | 0.62 | 1.18 | 2.51 | 5.01 |
| | NCI-H522 | 16.5 | 31.1 | 3.23 | 6.40 | 0.67 | 1.37 | 3.52 | 8.43 | 2.00 | 5.01 |
| Colon | COLO 205 | 16.8 | 32.8 | 4.15 | 14.20 | 0.85 | 1.86 | 0.93 | 2.71 | 2.00 | 3.16 |
| Cancer | HCC-2998 | 18.0 | 34.6 | 2.83 | 5.31 | 0.70 | 1.36 | 1.44 | 5.09 | 3.16 | NT^{c} |
| | HCT-116 | 4.09 | 75.6 | 1.12 | 4.83 | 0.69 | 1.35 | 0.64 | 1.30 | 1.58 | 2.51 |
| | HCT-15 | 2.07 | 4.33 | 0.33 | 0.70 | 0.70 | 1.41 | 0.67 | 1.35 | 2.51 | 3.16 |
| | HT29 | 1.92 | 4.19 | 0.53 | 2.11 | 0.67 | 1.37 | 0.67 | 1.32 | 2.00 | 5.01 |
| | KM12 | 17.4 | 43.3 | 3.25 | 6.65 | 0.80 | 1.66 | 1.01 | 2.91 | 1.58 | 2.51 |
| | SW-620 | 2.11 | 4.62 | 0.31 | 0.62 | 0.68 | 1.30 | 0.64 | 1.26 | 2.51 | 5.01 |
| CNS Cancer | SF-268 | 17.5 | 39.2 | 3.09 | 7.00 | 0.70 | 1.62 | 3.56 | 10.90 | 2.51 | 3.98 |
| | SF-295 | 18.0 | 36.9 | 2.06 | 4.47 | 0.57 | 1.13 | 0.58 | 1.14 | 1.58 | 6.31 |
| | SF-539 | 13.6 | 26.5 | 2.30 | 4.57 | 0.50 | 0.99 | 0.53 | 1.02 | 1.58 | 2.51 |
| | SNB-19 | 16.8 | 33.2 | 2.97 | 5.48 | 0.73 | 1.56 | 4.24 | 9.12 | 3.16 | 3.98 |
| | SNB-75 | 14.9 | 28.4 | 0.84 | 3.82 | 0.59 | 3.97 | 0.98 | 5.14 | 3.16 | 3.98 |
| | U251 | 3.43 | 14.7 | 2.69 | 5.53 | 0.67 | 1.34 | 1.24 | 4.82 | 2.00 | 3.16 |
| Melanoma | LOX IMVI | 0.66 | 24.5 | 0.67 | 2.66 | 0.62 | 1.15 | 0.56 | 1.05 | NT^{c} | NT^{c} |
| | MALME-3M | 1.68 | 3.21 | 0.29 | 0.53 | 0.64 | 1.18 | 0.58 | 1.05 | 2.00 | 39.81 |
| | M14 | 1.79 | 3.71 | 0.40 | 1.05 | 0.67 | 1.41 | 0.72 | 1.90 | 2.00 | 3.16 |
| | MDA-MB-435 | 1.75 | 3.27 | 0.27 | 0.56 | 0.62 | 1.26 | 0.65 | 1.31 | 1.58 | 5.01 |
| | SK-MEL-2 | 100 | 100 | 9.87 | >18.0 | 1.00 | 2.60 | 4.82 | 9.89 | 2.00 | 6.31 |
| | SK-MEL-28 | 1.67 | 3.11 | 0.32 | 0.58 | 0.64 | 1.17 | 0.75 | 1.85 | 2.51 | 2.51 |
| | SK-MEL-5 | 17.5 | 31.7 | 2.05 | 4.26 | 0.67 | 1.21 | 1.41 | 5.28 | 1.58 | 2.51 |
| | UACC-257 | 15.3 | 28.8 | 3.05 | 5.99 | 0.69 | 1.43 | 3.45 | 8.11 | 2.00 | 5.01 |
| | UACC-62 | 18.4 | 33.9 | 3.06 | 5.66 | 0.66 | 1.24 | 3.99 | 8.28 | 1.58 | 2.51 |
| Ovarian | IGROV1 | 17.7 | 36.6 | 3.01 | 5.61 | 0.74 | 1.39 | 1.42 | 5.11 | 2.51 | 10.00 |
| Cancer | OVCAR-3 | 9.59 | 25.8 | 2.54 | 5.06 | 0.65 | 1.33 | 0.64 | 1.36 | 3.16 | 3.98 |
| | OVCAR-4 | 15.7 | 31.6 | 2.63 | 5.63 | 0.74 | 1.62 | 0.88 | 2.45 | 3.16 | 25.12 |
| | OVCAR-5 | 15.6 | 29.0 | 2.82 | 5.23 | 0.65 | 1.23 | 1.60 | 6.09 | 3.16 | 6.31 |
| | OVCAR-8 | 17.4 | 35.4 | 3.85 | 10.50 | 0.84 | 1.95 | 5.69 | 17.5 | 3.16 | 12.59 |
| | NCI/ADR-RES | 14.2 | 45.4 | 1.78 | 8.09 | 1.17 | 3.71 | 3.66 | 10.00 | 2.51 | 7.94 |
| | SK-OV-3 | 18.5 | 34.7 | 3.48 | 7.50 | 2.93 | 8.23 | 5.33 | 1.08 | 2.51 | 5.01 |
| Renal | 786-0 | 1.81 | 3.52 | 0.30 | 0.58 | 0.53 | 1.10 | 0.50 | 1.03 | 3.16 | 5.01 |
| Cancer | A498 | 100 | 100 | 2.74 | 5.31 | 4.71 | 9.61 | 5.11 | 9.70 | 2.51 | 3.16 |
| | ACHN | 15.5 | 28.9 | 2.39 | 4.73 | 0.64 | 1.18 | 1.02 | 3.47 | 2.51 | 10.00 |
| | CAKI-1 | 16.1 | 29.6 | 3.01 | 5.60 | 0.65 | 1.22 | 0.80 | 2.08 | 3.16 | 12.59 |
| | RXF 393 | 3.38 | 15.0 | 0.49 | 1.66 | 0.51 | 1.11 | 0.65 | 1.73 | 3.16 | 5.01 |
| | SN12C | 16.7 | 34.7 | 2.77 | 5.30 | 0.68 | 1.26 | 0.67 | 1.38 | 2.51 | 3.16 |
| | TK-10 | 18.5 | 33.2 | 3.86 | 6.63 | 0.71 | 1.31 | 1.35 | 4.87 | 3.98 | 10.00 |
| | UO-31 | 15.3 | 29.2 | 2.71 | 5.14 | 0.59 | 1.11 | 2.18 | 6.81 | 2.51 | 6.31 |
| Prostate | PC-3 | 15.1 | 30.1 | 2.88 | 6.03 | 0.72 | 1.52 | 0.76 | 2.10 | 2.00 | 5.01 |
| Cancer | DU-145 | 17.6 | 35.6 | 2.23 | 4.90 | 0.72 | 1.35 | 0.75 | 1.58 | 3.16 | 7.94 |
| Breast | MCF7 | 1.67 | 3.45 | 0.29 | 0.61 | 0.65 | 1.30 | 0.63 | 1.50 | 2.51 | 7.94 |
| Cancer | MDA-MB- | 17.5 | 38.1 | 2.78 | 5.25 | 0.67 | 1.26 | 0.61 | 1.18 | 1.26 | 3.16 |
| | 231/ATCC | | | | | | | | | | |
| | HS 578T | 15.0 | 38.9 | 2.39 | 6.88 | 0.71 | 2.19 | 0.88 | 3.45 | 2.51 | 3.98 |
| | BT-549 | 16.5 | 35.3 | 2.78 | 5.67 | 2.10 | 7.41 | 4.83 | 9.57 | 3.16 | 3.98 |
| | T-47D | 17.0 | 43.4 | 3.72 | >18.0 | 7.62 | 24.7 | 1.93 | 8.74 | 1.58 | 5.01 |
| | MDA-MB-468 | 14.8 | 32.9 | 2.48 | 5.03 | 0.71 | 1.36 | 0.77 | 1.85 | 3.16 | 7.94 |

The target compounds were tested against hepatocellular carcinoma cell lines. Compounds 1j. 13a. 13b. 13c. 13e. and 13f showed higher inhibition activity than sorafenib and compound 1m showed inhibition activity comparable to sorafenib. The mean inhibition percentage values are illustrated in Table 9.

The mean inhibition percentage (IC50) values of sorafenib and compounds 1j. 1m. 13a. 13b. 13c. 13c. 13f against HepG2 and Hep3B HCC cell lines are shown in Table 10.

TABLE 10

IC₅₀ value (μM)^{a}

| Compound No. | HepG2 | Hep3B |
|---|---|---|
| 1j | 3.68 ± 0.22 | 3.87 ± 0.13 |
| 1m | 5.74 ± 0.20 | 6.56 ± 0.31 |
| 13a | 1.27 ± 0.14 | 1.89 ± 0.18 |
| 13b | 1.48 ± 0.09 | 2.32 ± 0.02 |
| 13c | 2.37 ± 0.04 | 1.79 ± 0.25 |
| 13e | 0.80 ± 0.04 | 1.76 ± 0.01 |
| 13f | 0.46 ± 0.06 | 1.09 ± 0.14 |
| Sorafenib | 2.86 ± 1.40 | 4.01 ± 0.83 |

^{a}Mean of triplicate assays ± SEM.

The compounds of the present disclosure and sorafenib were tested against a panel of 50 kinases (Tables 11-12). The results showed that the compounds are selective against FMS kinase, while sorafenib is a multikinase inhibitor as shown below. Table 11 shows the mean inhibition percentage values of compound 13b (1 μM concentration, duplicate assay, 10 UM ATP concentration) against a 50-kinase panel.

Table 12 shows the mean inhibition percentage values of compound 13c (1 μM concentration, duplicate assay, 10 μM ATPconcentration) against a 50-kinase panel.

Table 13 shows the mean inhibition percentage values of Sorafenib (1 μM concentration,

duplicate assay, 10 μM ATP concentration) against a 50-kinase panel.

Table 14 shows the mean inhibition percentage the IC₅₀ values of compounds 13b, 13c, and sorafenib against the most sensitive kinases. Compounds 13b and 13c showed high potency and selectivity against FMS kinase.

TABLE 14

| | IC₅₀ (nM)^{a} | | |
|---|---|---|---|
| Kinase | 13b | 13c | Sorafenib |
| Flt3 | 362.0 ± 0.4 | 483.6 ± 0.7 | 177.0 ± 0.5 |
| Flt4 | 366.3 ± 0.5 | 945.1 ± 0.5 | 762.3 ± 1.2 |
| FMS | 21.5 ± 0.2 | 73.9 ± 0.2 | 80.0 ± 0.7 |
| PDGFRα | 972.4 ± 1.3 | 992.3 ± 0.6 | 103.2 ± 0.9 |
| TrkA | NT | 958 ± 0.4 | 82.9 ± 1.0 |
| TrkC | NT | 889.2 ± 0.7 | 37.2 ± 0.4 |

^{a}The results are expressed as means of duplicate assays ± SEM.

### Synthesis of 2-(4-nitrophenyl)-1,5-naphthyridine-4-carboxylic acid (Compound 4)

To a solution of compound 4-Azaisatin 2 (500 mg, 3.38 mmol) dissolved in ethanol (10 mL), K₂CO₃ (284.5 mg, 5.07 mmol) dissolved in water (1 mL) was added and the mixture was stirred for 30 minutes at room temperature. After complete hydrolysis of compound 2 was detected by LC-MS, compound 3 (558.2 mg, 3.38 mmol) dissolved in ethanol (10 mL), was added dropwise and the reaction mixture was continuously stirred at room temperature for 2 h. Lastly, another one equivalent of KOH (189.6 mg, 3.38 mmol) was added to the mixture and stirred overnight at room temperature. After completion of reaction which was detected by TLC and LC-MS, EtOH in the reaction mixture was evaporated and 1M HCl was added dropwise to achieve moderately acidic pH (identified by change in color of pH paper to yellow) then the yellow solid was filtered and washed with minimal amount of water. The filtrate was dried under reduced pressure. Purification was done by normal phase column chromatography, mobile phase: ethyl acetate/hexane 10:90 v/v followed by ethyl acetate/hexane 80:20 v/v; Spectral data of the target compounds: Yield: 89%; ¹H NMR (DMSO-d₆, 500 MHz) δ 9.01 (d, J=2.5 Hz, 1H), 8.57-8.55 (m, 2H), 8.51 (dd, J=1.0 Hz, 8.5 Hz, 1H), 8.41-8.38 (m, 2H), 8.30 (brs, 1H), 7.84 (dd, J=4.0 Hz, 8.5 Hz, 1H); ¹³C NMR (DMSO-d₆, 125 MHz) δ166.7. 155.0, 151.7, 148.5, 143.3, 143.2. 139.1. 138.3, 129.6, 128.9, 126.2, 124.1, 123.8, 123.7, 120.9; MS m/z: 296.05 [M+1]⁺

### General Synthesis and Spectral Data of Derivatives 5a-c

Compound 4 (150 mg, 0.51 mmol) was dissolved in DMF (4 mL) at −15° C. under N_{2 (g)} and TEA (142 μL) was added thereto dropwisely. The reaction mixture was stirred for 15 min. TBTU (327.5 mg. 1.02 mmol) was added and stirred for 30 min while maintaining temperature −15° C. under N_{2 (g)}. 5 equivalent of TEA (355 μL) was added simultaneously with the addition of appropriate amine (2.55 mmol) and stirred for 2 h under N_{2 (g)}. The crude compound was washed with ice-cooled H₂O, and the precipitate was filtered via vacuum filtration. The filtrate was dried in vacuum desiccator, and purified by column chromatography (silica gel, hexane/ethyl acetate). MS m/z: 309.34 [M+1]⁺

### N-Methyl-2-(4-nitrophenyl)-1,5-naphthyridine-4-carboxamide (Compound 5a)

Purification done by normal phase column chromatography, mobile phase: ethyl acetate/hexane 10:90 v/v followed by ethyl acetate/hexane 45:55 v/v; Spectral data of the target compounds: Yield: 77%; ¹H NMR (CDC13, 500 MHz) δ10.97 (brs, 1H), 9.17 (brs, 1H), 9.02 (dd. J=1.5 Hz, 4.0 Hz, 1H), 8.63 (dd, J=1.5 Hz, 8.5 Hz, 1H), 8.48 (dd, J=2.0 Hz, J=7.0 Hz, 2H), 8.41 (dd, J=2.0 Hz, 7.0 Hz, 2H), 7.79 (dd, J=4.0 Hz, 8.5 Hz, 1H), 3.20 (d. J=4.5 Hz, 3H); ¹³C NMR (CDCl₃, 125 MHz) δ 164.6, 156.1, 150.6, 149.0, 144.5, 143.9, 140.5, 139.5, 137.2, 128.8, 124.9, 124.3, 124.1, 26.9; MS m/z: 308.78 [M+1]⁺.

### N-Isopropyl-2-(4-nitrophenyl)-1,5-naphthyridine-4-carboxamide (compound 5b)

Purification done by normal phase column chromatography, mobile phase: ethyl acetate/hexane 10:90 v/v followed by ethyl acetate/hexane 38:62 v/v; Spectral data of the target compounds: Yield: 73%; ¹H NMR (Acetone-d₆, 500 MHz) δ10.79 (d, J=4.5 Hz, 1H), 9.15 (dd, J=2.0 Hz, 4.5 Hz, 1H), 9.08 (brs, 1H), 8.68 (dd, J=1.5 Hz, 8.5 Hz, 1H), 8.63-8.60 (m, 2H), 8.47-8.44 (m, 2H), 7.97 (dd, J=4.5 Hz, J=8.5 Hz, 1H), 4.36-4.29 (m, 1H), 1.36 (brs, 6H); ¹³C NMR(Acetone-d₆, 125 MHz) δ162.9, 156.4, 152.1, 149.9, 145.2, 144.8, 1421.1, 140.1, 138.7, 129.6, 126.2, 124.9, 124.1, 42.7, 22.9; MS m/z: 337.05 [M+1]⁺

### 2-(4-Nitrophenyl)-N-(pyridin-2-yl)-1,5-naphthyridine-4-carboxamide (Compound 5c)

Purification done by normal phase column chromatography, mobile phase: ethyl acetate/hexane 10:90 v/v followed by ethyl acetate/hexane 40:60 v/v; Spectral data of the target compounds: Yield: 63%; ¹H NMR (CDC13, 500 MHz) δ13.91 (brs, 1H), 9.26 (d, J=2.5 Hz, 9.22 (brs, 1H), 8.67 (dd, J=1.5 Hz, 8.5 Hz, 1H), 8.56 (d, J=8.0 Hz, 1H), 8.50-8.47 (m, 3H), 8.42 (d, J=9.0 Hz, 2H), 7.86 (dd, J=4.5 Hz, 8.5 Hz, 2H), 7.17 (dd, J=5.0 Hz, 6.5 Hz, 1H); ¹³C NMR (CDCl₃, 125 MHz) δ162.3, 156.1, 151.2, 149.1, 144.7, 143.8, 140.1, 139.6, 136.5, 128.8, 125.3, 124.4, 124.2, 120.5, 115.6.

### General Synthesis of Derivatives 6a-c

To the solution of compound 5a-c (0.49 mmol) in MeOH/H₂O [1:1 v/v] (2.5 mL of each) was added NH₄Cl (4.9 mmol) and Fe powder (137 mg, 2.45 mmol), stirred for 15 min at room temperature under N_{2 (g)}. The mixture was then heated at 80° C. overnight. The crude compound was dried under reduced pressure to remove excess methanol and the crude compound was filtered using ethylacetate. The filtrate was dried in vacuum desiccator, and purified by column chromatography (silica gel, hexane/ethyl acetate)

### 2-(4-Aminophenyl)-N-methyl-1,5-naphthyridine-4-carboxamide (Compound 6a)

Purification done by normal phase column chromatography, mobile phase: ethyl acetate/hexane 10:90 v/v followed by ethyl acetate/hexane 62:38 v/v; Spectral data of the target compounds: Yield: 54%; ¹H NMR (DMSO-d₆, 500 MHz) δ10.27 (d, J=4.5 Hz, 1H), 8.94 (d, J=4.0 Hz, 1H), 8.64 (brs, 1H), 8.46 (d, J=8.5 Hz, 1H), 8.04 (d, J=8.5 Hz, 2H), 7.83 (dd, J=4.0 Hz, 8.5 Hz, 1H), 6.71 (dd, J=8.5 Hz, 2H), 5.75 (brs, 2H), 2.99 (d, J=4.5 Hz, 3H); MS m/z: 279.16 [M+1]⁺.

2-(4-Aminophenyl)-N-isopropyl-1,5-naphthyridine-4-carboxamide (compound 6b)

Purification done by normal phase column chromatography, mobile phase: ethyl acetate/hexane 10:90 v/v followed by ethyl acetate/hexane 55:45 v/v; Spectral data of the target compounds: Yield: 58%; ¹H NMR (Acetone-d₆, 500 MHz) δ10.89 (brs, 1H), 8.95 (d, J=6.0 Hz, 2H), 8.49 (d, J=8.5 Hz, 1H), 8.14 (d, J=8.0 Hz, 2H), 7.82 (dd, J=4.0 Hz, J=8.5 Hz, 1H), 6.89-6.84 (m, 2H), 4.31 (dd, J=6.5 Hz, 13.5 Hz, 1H), 1.35 (d, J=6.5 Hz, 6H); ¹³C NMR(Acetone-d₆, 125 MHz) δ 163.5, 159.0, 152.0, 149.6, 145.3, 140.7, 139.6, 139.1, 137.3, 129.7, 129.2, 127.0, 125.7, 125.5, 123.5, 123.1, 120.9, 115.1, 42.6, 22.9.

### 2-(4-Aminophenyl)-N-(pyridin-2-yl)-1,5-naphthyridine-4-carboxamide (Compound 6c)

Purification done by normal phase column chromatography, mobile phase: ethyl acetate/hexane 10:90 v/v followed by ethyl acetate/hexane 65:35 v/v; Spectral data of the target compounds: Yield: 41%; ¹H NMR (DMSO-d₆, 500 MHz) δ13.43 (brs, 1H), 9.06 (dd, J=1.0 Hz, 4.0 Hz, 1H), 8.85 (brs, 1H), 8.54 (dd, J=1.5 Hz, 8.5 Hz, 1H), 8.44 (d, J=4.0 Hz, 1H), 8.39 (d, J=8.5 Hz, 1H), 8.08 (d, J=8.5 Hz, 2H), 7.94-7.89 (m, 2H), 7.23 (dd, J=5.0 Hz, 6.5 Hz, 1H), 6.75 (d, J=8.5 Hz, 2H); ¹³C NMR (DMSO-d₆, 125 MHz) δ 158.5, 149.3, 149.1, 139.6, 138.7, 129.4, 129.1, 125.5, 124.8, 124.0, 123.9, 120.2, 115.9, 115.3; MS m/z: 342.21 [M+1]⁺.

### General Synthesis of Derivatives 1a-m

A mixure of compound 6a-c (0.15 mmol) and the appropriate isocyanate/isothiocyanate (0.23 mmol) in DMF (2 mL) was stirred at 0° C. under N_{2 (g)}. The reaction mixture was stirred at room temperature overnight. After that, it was extracted by ethyl acetate (3×10 mL) and ice-cooled distilled water (30 mL). The organic layer was collected, dried over anhydrous Na₂SO₄ and was dried under reduced pressure.

### 2-(4-(3-(4-Fluorophenyl)ureido)phenyl)-N-methyl-1,5-naphthyridine-4-carboxamide (Compound 1a)

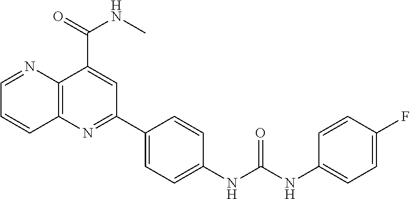 [SMILES: CNC(=O)c1cc(-c2ccc(NC(=O)Nc3ccc(F)cc3)cc2)nc2cccnc12]

Purification done by normal phase column chromatography, mobile phase: ethyl acetate/hexane 10:90 v/v followed by ethyl acetate/hexane 50:50 v/v; Spectral data of the target compounds: Yield: 41%; ¹H NMR (DMSO-d₆, 500 MHz) δ10.19 (d, J=4.5 Hz, 1H), 9.04-9.01 (m, 2H), 8.81 (brs, 1H), 8.78 (brs, 1H), 8.56 (dd, J=1.0 Hz, 7.0 Hz, 1H), 8.26 (d, J=10.0 Hz, 2H), 7.89 (dd, J=4.0 Hz, 4.5 Hz, 1H), 7.67 (d, J=8.5 Hz, 2H), 7.51-7.48 (m, 2H), 7.14 (d, J=9.0 Hz, 2H), 3.00 (d, J=4.5 Hz, 3H); ¹³C NMR (DMSO-d₆, 125 MHz) δ164.6, 158.5, 156.7, 156.6, 152.5, 150.3, 143.7, 142.0, 139.2-138.0 (J=62.5 Hz), 135.8, 130.8, 128.2, 125.4, 121.4, 120.3, 120.2, 118.3, 115.5, 115.3, 26.4; MS m/z: 416.24 [M+1]⁺.

### 2-(4-(3-(3,4-Dichlorophenyl)ureido)phenyl)-N-methyl-1,5-naphthyridine-4-carboxamide (Compound 1b)

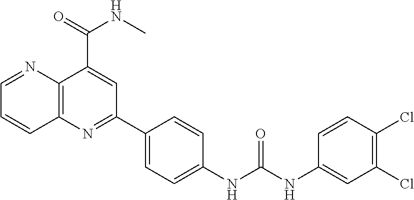 [SMILES: CNC(=O)c1cc(-c2ccc(NC(=O)Nc3ccc(Cl)c(Cl)c3)cc2)nc2cccnc12]

Purification done by normal phase column chromatography, mobile phase: ethyl acetate/hexane 10:90 v/v followed by ethyl acetate/hexane 30:70 v/v; Spectral data of the target compounds: Yield: 56%; ¹H NMR (DMSO-d₆, 500 MHz) δ10.18 (brs, 1H), 9.14 (d, J=28.5 Hz, 2H), 9.03 (brs, 1H), 8.74 (brs, 1H), 8.56 (d, J=10.0 Hz, 1H), 8.27 (d, J=10.0 Hz, 2H), 7.91 (brs, 2H), 7.69 (d, J=5.0 Hz, 2H), 7.54 (d, J=8.0 Hz, 1H), 7.36 (d, J=8.0 Hz, 1H), 3.01 (brs, 3H); ¹³C NMR (DMSO-d₆, 125 MHz) δ164.5, 156.6, 152.2, 150.4, 143.7, 141.6, 139.8, 139.2, 138.8, 138.0, 131.1, 131.1, 130.6, 128.2, 125.3, 123.4, 121.4, 119.5, 118.5, 118.5, 26.3; MS m/z: 268.32 [M+1]⁺.

### 2-(4-(3-(4-Fluoro-3-(trifluoromethyl)phenyl)ureido)phenyl)-N-methyl-1,5-naphthyridine-4-carboxamide (Compound 1c)

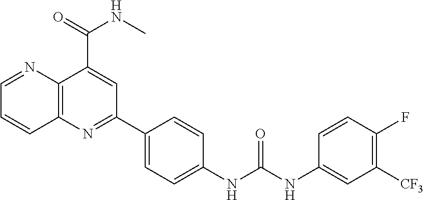 [SMILES: CNC(=O)c1cc(-c2ccc(NC(=O)Nc3ccc(F)c(C(F)(F)F)c3)cc2)nc2cccnc12]

Purification done by normal phase column chromatography, mobile phase: ethyl acetate/hexane 10:90 v/v followed by ethyl acetate/hexane 35:65 v/v; Spectral data of the target compounds: Yield: 53%; ¹H NMR (DMSO-d₆, 500 MHz) δ10.19 (d, J=4.5 Hz, 1H), 9.14 (d, J=9.5 Hz, 2H), 9.03 (dd, J=3.0 Hz, 4.5 Hz , 1H), 8.74 (brs, 1H), 8.56 (dd, J=1.5 Hz, 7.0 Hz, 1H), 8.27 (d, J=9.0 Hz, 2H), 8.04 (dd, J=2.0 Hz, 6.0 Hz, 1H), 7.89 (dd, J=4.5 Hz, 8.5 Hz, 1H), 7.70-7.66 (m, 3H), 7.45 (t, J=10.0 Hz, 1H), 3.00 (d, J=4.5 Hz, 3H); ¹³C NMR (DMSO-d₆, 125 MHz) δ164.5, 156.7, 152.4, 150.4, 143.7, 141.7, 139.2-138.0 (J=125.0 Hz), 136.4-136.3 (J=2.5 Hz), 131.1, 128.2, 125.3, 124.5, 124.5, 121.4, 118.5, 117.7, 117.6, 116.2, 116.2, 26.3; MS m/z: 484.33 [M+1]⁺.

### 2-(4-(3-(3-Chloro-4-(trifluoromethyl)phenyl)ureido)phenyl)-N-methyl-1,5-naphthyridine-4-carboxamide (Compound 1d)

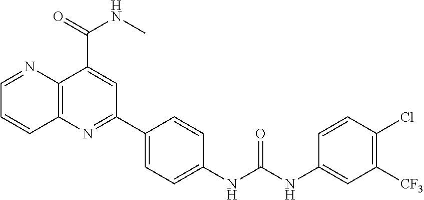 [SMILES: CNC(=O)c1cc(-c2ccc(NC(=O)Nc3ccc(Cl)c(C(F)(F)F)c3)cc2)nc2cccnc12]

Purification done by normal phase column chromatography, mobile phase: ethyl acetate/hexane 10:90 v/v followed by ethyl acetate/hexane 41:59 v/v; Spectral data of the target compounds: Yield: 48%; ¹H NMR (DMSO-d₆, 500 MHz) δ10.19 (d, J=4.5 Hz, 1H), 9.25 (brs, 1H), 9.18 (brs, 1H), 9.03 (dd, J=1.5 Hz, 4.0 Hz, 1H), 8.74 (brs, 1H), 8.56 (dd, J=2.0 Hz, 8.5 Hz, 1H), 8.27 (d, J=8.5 Hz, 2H), 8.14 (d, J=2.5 Hz, 1H), 7.89 (dd, J=4.5 Hz, 8.5 Hz, 1H), 7.70-7.62 (m, 4H), 3.00 (d, J=5.0 Hz, 3H); ¹³C NMR (DMSO-d₆, 125 MHz) δ164.5, 156.6, 152.3, 150.4, 143.7, 141.5, 139.2, 139.2, 138.7, 138.0, 132.1, 131.2, 128.2, 126.9-126.1 (J=30.5 Hz), 125.3, 123.9, 123.3, 122.6, 121.8, 121.4, 118.6, 117.0, 116.9, 116.8, 26.3; MS m/z: 500.33 [M+1]⁺.

### 2-(4-(3-(3,5-Bis(trifluoromethyl)phenyl)ureido)phenyl)-N-methyl-1,5-naphthyridine-4-carboxamide (Compound 1e)

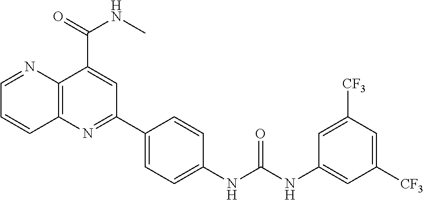 [SMILES: CNC(=O)c1cc(-c2ccc(NC(=O)Nc3cc(C(F)(F)F)cc(C(F)(F)F)c3)cc2)nc2cccnc12]

Purification done by normal phase column chromatography, mobile phase: ethyl acetate/hexane 10:90 v/v followed by ethyl acetate/hexane 48:52 v/v; Spectral data of the target compounds: Yield: 56%; ¹H NMR (DMSO-d₆, 500 MHz) δ10.19 (d, J=5.0 Hz, 1H), 9.47 (brs, 1H), 9.32 (brs, 1H), 9.03 (dd, J=1.5 Hz, 4.0 Hz, 1H), 8.74 (brs, 1H), 8.56 (dd, J=1.5 Hz, J=8.5 Hz, 1H), 8.28 (dd, J=1.5 Hz, 8.5 Hz, 2H), 8.16 (brs, 2H), 7.89 (dd, J=4.5 Hz, 8.5 Hz, 1H), 7.72 (d, J=8.5 Hz, 2H), 7.66 (brs, 1H), 3.00 (d, J=5.0 Hz, 3H); ¹³C NMR (DMSO-d₆, 125 MHz) δ164.5, 156.6, 152.3, 150.4, 143.7, 141.7, 141.3, 139.2, 138.7, 138.0, 131.4, 131.1-130.4 (J=129.5 Hz), 128.2, 126.6, 125.3, 124.4, 122.2, 121.4, 120.1, 118.8, 118.2, 114.6, 26.3; MS m/z: 534.23 [M+1]⁺.

### 2-(4-(3-(4-Fluorophenyl)ureido)phenyl)-N-isopropyl-1,5-naphthyridine-4-carboxamide (Compound 1f)

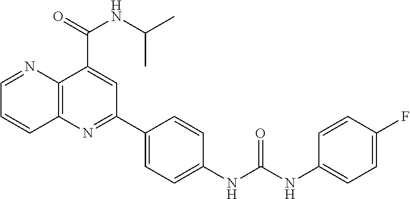 [SMILES: CC(C)NC(=O)c1cc(-c2ccc(NC(=O)Nc3ccc(F)cc3)cc2)nc2cccnc12]

Purification done by normal phase column chromatography, mobile phase: ethyl acetate/hexane 10:90 v/v followed by ethyl acetate/hexane 48:52 v/v; Spectral data of the target compounds: Yield: 56%; ¹H NMR (DMSO-d₆, 500 MHz) δ10.28 (d, J=7.0 Hz, 1H), 9.04 (d, J=1.5 Hz, 4.0 Hz, 1H), 9.00 (brs, 1H), 8.80 (brs, 1H), 8.73 (brs, 1H), 8.56 (dd, J=1.5 Hz, 8.5 Hz, 1H), 8.27 (d, J=9.0 Hz, 2H), 7.89 (dd, J=4.0 Hz, 8.5 Hz, 1H), 7.68 (d, J=8.5 Hz, 2H), 7.51-7.48 (m, 2H), 7.16-7.09 (m, 2H), 4.22 (dd, J=7.0 Hz, 14.0 Hz, 1H), 1.30 (d, J=7.0 Hz, 6H); ¹³C NMR (DMSO-d₆, 125 MHz) δ162.9, 158.4, 156.7, 156.5, 152.4, 150.3, 143.6, 142.0, 139.3, 138.6, 138.1, 136.0-135.8 (J=25.5 Hz), 130.7, 128.2, 125.3, 121.4, 120.2, 120.1, 120.0, 120.0, 118.2, 115.4, 115.3, 115.2, 115.2, 41.4, 22.5; MS m/z: 444.25 [M+1]⁺.

### 2-(4-(3-(4-Fluoro-3-(trifluoromethyl)phenyl)ureido)phenyl)-N-isopropyl-1,5-naphthyridine-4-carboxamide (Compound 1g)

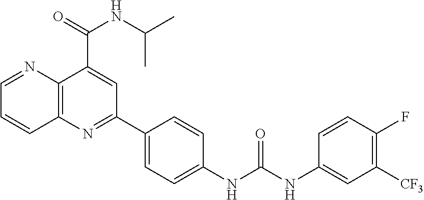 [SMILES: CC(C)NC(=O)c1cc(-c2ccc(NC(=O)Nc3ccc(F)c(C(F)(F)F)c3)cc2)nc2cccnc12]

Purification done by normal phase column chromatography, mobile phase: ethyl acetate/hexane 10:90 v/v followed by ethyl acetate/hexane 38:62v/v; Spectral data of the target compounds: Yield: 67%; ¹H NMR (DMSO-d₆, 500 MHz) δ10.29 (d, J=7.5 Hz, 1H), 9.13 (d, J=10.5 Hz, 2H), 9.03 (dd, J=2.0 Hz, 4.5 Hz, 1H), 8.73 (brs, 1H), 8.56 (dd, J=1.5 Hz, 8.5 Hz, 1H), 8.27 (d, J=8.5 Hz, 2H), 8.03 (dd, J=2.5 Hz, 6.5, 1H), 7.88 (dd, J=4.0 Hz, 8.5 Hz, 1H), 7.70-7.65 (m, 3H), 7.45 (t, J=10.0 Hz, 1H), 4.22 (dd, J=6.5 Hz, 13.5 Hz, 1H), 1.30 (d, J=6.5 Hz, 6H) ;-¹³C NMR (DMSO-d₆, 125 MHZ) δ162.9, 156.6, 152.4, 150.3, 143.6, 141.6, 139.3, 138.6, 138.1, 136.4-136.3 (J=12.5 Hz), 131.1, 128.1, 125.3, 124.5, 124.4, 121.4, 118.5, 117.7, 117.7-116.1 (J=25.0 Hz), 41.4, 22.4; MS m/z: 512.24 [M+1]⁺.

### 2-(4-(3-(3,5-Bis(trifluoromethyl)phenyl)ureido)phenyl)-N-isopropyl-1,5-naphthyridine-4-carboxamide (Compound 1h)

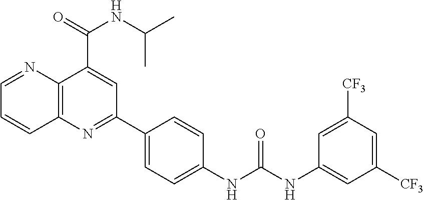 [SMILES: CC(C)NC(=O)c1cc(-c2ccc(NC(=O)Nc3cc(C(F)(F)F)cc(C(F)(F)F)c3)cc2)nc2cccnc12]

Purification done by normal phase column chromatography, mobile phase: ethyl acetate/hexane 10:90 v/v followed by ethyl acetate/hexane 40:60v/v; Spectral data of the target compounds: Yield: 64%; ¹H NMR (DMSO-d₆, 500 MHz) δ9.83(d, J=7.0 Hz, 1H), 9.03 (brs, 1H), 8.88 (brs, 1H), 8.59 (dd, J=2.0 Hz, 4.5 Hz, 1H), 8.29 (brs, 1H), 8.12 (dd, J=1.5 Hz, 8.5 Hz, 1H), 7.84 (d, J=8.5 Hz, 2H), 7.18 (brs, 2H), 7.44 (dd, J=4.0 Hz, 8.5 Hz, 1H) 7.27 (d, J=8.5 Hz, 2H), 7.22 (brs, 1H), 3.77 (dd, J=7.0 Hz, 14.0 Hz, 1H), 0.85 (d, J=6.5 Hz, 6H); ¹³C NMR (DMSO-d₆, 125 MHz) δ163.0, 156.6, 152.3, 150.4, 143.7, 141.7, 141.3, 139.3, 138.7, 138.1, 131.4, 131.1-130.4 (J=32.5 Hz), 128.2, 126.6, 125.3, 124.4, 122.3, 121.4, 120.1, 118.8, 118.2, 114.7, 41.4, 22.5; MS m/z: 562.27 [M+1]⁺.

### 2-(4-(3-(4-Fluorophenyl)ureido)phenyl)-N-(pyridin-2-yl)-1,5-naphthyridine-4-carboxamide (Compound 1i)

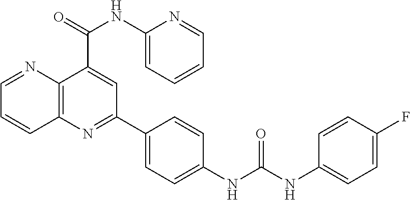 [SMILES: O=C(Nc1ccc(F)cc1)Nc1ccc(-c2cc(C(=O)Nc3ccccn3)c3ncccc3n2)cc1]

Purification done by normal phase column chromatography, mobile phase: ethyl acetate/hexane 10:90 v/v followed by ethyl acetate/hexane 65:35v/v; Spectral data of the target compounds: Yield: 34%; ¹H NMR (DMSO-d₆, 500 MHz) δ13.33 (brs, 1H), 9.14 (dd, J=2.0 Hz, 4.5 Hz, 1H), 9.03(brs, 1H), 8.94(brs, 1H), 8.81(brs, 1H), 8.64 (dd, J=1.5 Hz, 7.0 Hz, 1H), 8.45 (dd, J=1.0 Hz, 4.5 Hz, 1H), 8.41 (d, J=8.5 Hz 1H), 8.30 (d, J=8.5, 2H), 7.98-7.92 (m, 2H), 7.70-7.69, 7.51-7.49 (m, 2H), 7.25-7.23(m, 1H), 7.15 (dd, J=2.0 Hz, 8.5 Hz, 2H); ¹³C NMR (DMSO-d₆, 125 MHZ) δ156.9, 156.6, 152.4, 151.5, 150.3, 148.5, 143.8, 142.2, 139.0, 138.7, 138.6, 136.8, 135.8, 135.8, 130.6, 128.3, 125.6, 122.2, 120.4-120.2 (J=19.5 Hz), 120.2, 118.3, 115.4, 115.3, 114.1; MS m/z: 479.21 [M+1]⁺.

### 2-(4-(3-(4-Fluoro-3-(trifluoromethyl)phenyl)ureido)phenyl)-N-(pyridin-2-yl)-1,5-naphthyridine-4-carboxamide (Compound 1j)

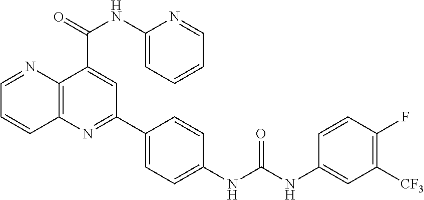 [SMILES: O=C(Nc1ccc(-c2cc(C(=O)Nc3ccccn3)c3ncccc3n2)cc1)Nc1ccc(F)c(C(F)(F)F)c1]

Purification done by normal phase column chromatography, mobile phase: ethyl acetate/hexane 10:90 v/v followed by ethyl acetate/hexane 56:44 v/v; Spectral data of the target compounds: Yield: 42%; ¹H NMR (DMSO-d₆, 500 MHz) δ13.24 (brs, 1H), 9.15-9.12 (m, 3H), 8.94 (brs, 1H), 8.64 (dd, J=1.5 Hz, 8.5 Hz, 1H), 8.44 (dd, J=1.0 Hz, 5.0 Hz, 1H), 8.40 (d, J=8.5 Hz, 1H), 8.30 (d, J=8.5 Hz, 2H), 8.04 (dd, J=3.0 Hz, 6.5 Hz, 1H), 7.98-7.92 (m, 2H), 7.72-7.66 (m, 3H), 7.45 (t, J=10.0 Hz, 1H), 7.25-7.22 (m, 1H); ¹³C NMR (DMSO-d₆, 125 MHz) δ149.4, 148.5, 147.3, 145.5, 140.8 138.8, 135.9, 135.6, 135.6, 133.7 133.4 133.4, 127.9, 125.2, 122.9 122.6, 120.7, 119.2, 118.5, 117.4, 115.5 114.7, 114.5, 113.6, 113.5, 113.2 (J=19.0 Hz), 111.1; MS m/z: 547.32 [M+1]⁺.

### 2-(4-(3-(3,5-Bis(trifluoromethyl)phenyl)ureido)phenyl)-N-(pyridin-2-yl)-1,5-naphthyridine-4-carboxamide (Compound 1k)

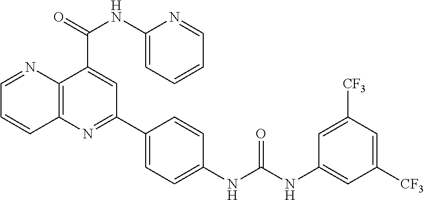 [SMILES: O=C(Nc1ccc(-c2cc(C(=O)Nc3ccccn3)c3ncccc3n2)cc1)Nc1cc(C(F)(F)F)cc(C(F)(F)F)c1]

Purification done by normal phase column chromatography, mobile phase: ethyl acetate/hexane 10:90 v/v followed by ethyl acetate/hexane 52:48 v/v; Spectral data of the target compounds: Yield: 42%; ¹H NMR (CDCl₃-DMSO-d₆, 500 MHz) δ13.96 (brs, 1H), 9.22 (brs, 1H), 9.06 (dd, J=1.5 Hz, 4.5 Hz, 1H), 8.99 (brs, 1H), 8.82 (brs, 1H), 8.52 (dd, J=2.0 Hz, 8.5 Hz, 1H), 8.42-8.37 (m, 2H), 8.18 (d, J=8.5 Hz, 2H), 7.98 (brs, 2H), 7.84-7.80 (m, 1H), 7.76 (dd, J=4.0 Hz, 8.5 Hz, 1H), 7.63 (d, J=8.5 Hz, 2H), 7.73(brs, 1H), 7.14-7.11 (m, 1H); ¹³C NMR (DMSO-d₆, 125 MHz) δ162.1, 157.1, 152.0, 150.7, 149.1, 146.7, 143.8, 141.0, 141.0, 138.9, 138.8, 1238.5, 134.8, 131.6-130.8 (J=34.5 Hz), 127.9, 126.1, 124.4, 124.0, 123.0, 121.8, 119.8, 119.6, 118.3, 117.6, 114.8, 114.3; MS m/z: 597.24 [M+1]⁺.

### 2-(4-(3-(4-Chloro-3-(trifluoromethyl)phenyl)thioureido)phenyl)-N-methyl-1,5-naphthyridine-4-carboxamide (Compound 1l)

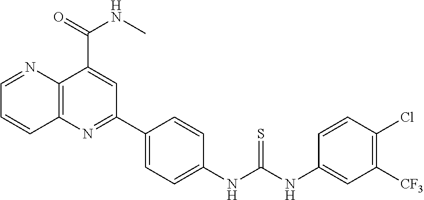 [SMILES: CNC(=O)c1cc(-c2ccc(NC(=S)Nc3ccc(Cl)c(C(F)(F)F)c3)cc2)nc2cccnc12]

Purification done by normal phase column chromatography, mobile phase: ethyl acetate/hexane 10:90 v/v followed by ethyl acetate/hexane 75:25 v/v; Spectral data of the target compounds: Yield: 47%; ¹H NMR (DMSO-d₆, 500 MHz) δ10.37((brs, 1H), 10.24 (brs, 1H), 10.16 (d, J=5.0 Hz, 1H), 9.06 (dd, J=1.5 Hz, 4.0 Hz, 1H), 8.77 (brs, 1H), 8.59 (dd, J=1.5 Hz, 8.5 Hz, 1H), 8.31 (dd, J=2.0 Hz, 7.0 Hz, 2H), 8.12 (d, J=2.5 Hz, 1H), 7.92 (dd, J=4.0 Hz, 4.5 Hz, 1H), 7.84 (dd, J=2.5 Hz, 9.0 Hz, 1H), 7.71 (dd, J=9.0 Hz, 15.0 Hz, 3H), 3.00 (d, J=5.0 Hz, 3H); ¹³C NMR (DMSO-d₆, 125 MHz) δ179.6, 164.4, 156.5, 150.7, 143.6, 141.1, 139.3, 139.1, 139.0, 138.1, 133.5, 131.6, 128.5, 127.8, 126.6-126.1 (J=31.5 Hz), 125.4, 125.3, 123.8, 123.4, 122.4, 122.4, 122.4, 121.6, 121.5, 26.3; MS m/z: 516.21 [M+1]⁺.

### 2-(4-(3-(3,5-Bis(trifluoromethyl)phenyl)thioureido)phenyl)-N-methyl-1,5-naphthyridine-4-carboxamide (Compound 1m)

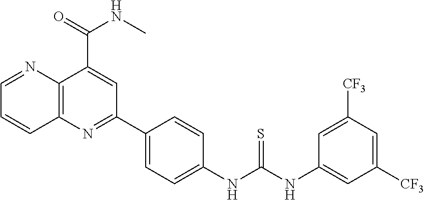 [SMILES: CNC(=O)c1cc(-c2ccc(NC(=S)Nc3cc(C(F)(F)F)cc(C(F)(F)F)c3)cc2)nc2cccnc12]

Purification done by normal phase column chromatography, mobile phase: ethyl acetate/hexane 10:90 v/v followed by ethyl acetate/hexane 70:30 v/v; Spectral data of the target compounds: Yield: 45%;¹H NMR (DMSO-d₆, 500 MHz) δ10.54 (brs, 1H), 10.39 (brs, 1H), 10.18 (d, J=4.5 Hz, 1H), 9.06 (d, J=2.5 Hz, 1H), 8.77 (brs, 1H), 8.58 (d, J=7.5 Hz, 1H), 8.31 (t, J=8.5 Hz, 4H), 7.90 (dd, J=4.5 Hz, J=8.5 Hz, 1H), 7.82 (brs, 1H), 7.72 (d, J=8.5, 2H), 3.00 (d, J=5.0 Hz, 3H); ¹³C NMR (DMSO-d₆, 125 MHz) δ179.7, 164.4, 156.5, 150.7, 143.7, 141.7, 140.8, 139.3, 138.9, 138.2, 133.8, 130.5-129.7 (J=32.5 Hz), 128.0, 126.5, 125.4, 124.3, 123.7, 123.6, 122.2, 121.6, 120.0, 117.1, 26.3; MS m/z: 550.11 [M+1]⁺.

### General Synthesis of Derivatives 1n-q

A mixture of appropriate amine 6a-c (0.15 mmol) and appropriate sulfonyl chloride (0.16 mmol) in pyridine (2 mL) was stirred for 15 min at 0° C. The reaction was stirred at room temperature overnight. After completion, the organic solvent was evaporated under reduced pressure and then mixture was extracted by DCM (3×10 mL) and ice-cooled distilled water (20 mL). The organic layer was collected, dried over anhydrous Na₂SO₄, and was dried under reduced pressure.

### 2-(4-((4-Fluorophenyl)sulfonamido)phenyl)-N-methyl-1,5-naphthyridine-4-carboxamide (Compound 1n)

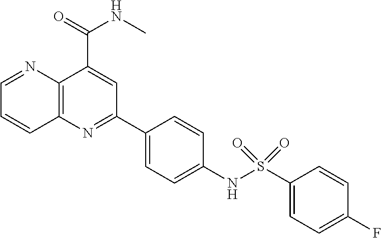 [SMILES: CNC(=O)c1cc(-c2ccc(NS(=O)(=O)c3ccc(F)cc3)cc2)nc2cccnc12]

Purification done by normal phase column chromatography, mobile phase: ethyl acetate/hexane 10:90 v/v followed by ethyl acetate/hexane 85:15 v/v; Spectral data of the target compounds: Yield: 21%; ¹H NMR (DMSO-d₆, 500 MHz) δ10.25 (brs, 1H), 9.68 (d, J=4.5 Hz, 1H), 8.59 (dd, J=2.0 Hz, 4.5 Hz, 1H), 8.21 (brs, 1H), 8.09 (dd, J=1.5 Hz, 8.5 Hz, 1H), 7.74 (dd, J=1.5 Hz, 6.5 Hz, 2H), 7.46-7.43 (m, 3H), 6.99-6.95 (m, 2H), 6.86 (dd, J=2.0 Hz, 7.0 Hz, 2H), 2.55 (dd, J=5.0 Hz, 13.5 Hz, 3H); MS m/z: 437.19 [M+1]⁺.

### N-Methyl-2-(4-((4-methylphenyl)sulfonamido)phenyl)-1,5-naphthyridine-4-carboxamide (Compound 1o)

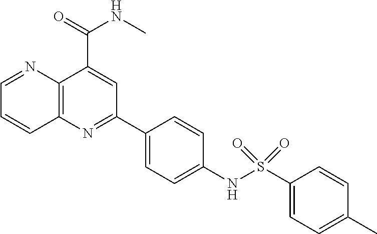 [SMILES: CNC(=O)c1cc(-c2ccc(NS(=O)(=O)c3ccc(C)cc3)cc2)nc2cccnc12]

Purification done by normal phase column chromatography, mobile phase: ethyl acetate/hexane 10:90 v/v followed by ethyl acetate/hexane 70:30 v/v; Spectral data of the target compounds: Yield: 54%; ¹H NMR (DMSO-d₆, 500 MHz) δ 10.61 (brs, 1H), 10.12 (d, J=4.5 Hz, 1H), 9.04 (dd, J=1.5 Hz, 4.0 Hz, 1H), 8.66 (brs, 1H), 8.53 (dd, J=1.5 Hz, 8.5 Hz, 1H), 8.17 (t, J=7.0 Hz, 2H), 7.89 (dd, J=4.5 Hz, 8.5 Hz, 1H), 7.73 (d, J=8.0 Hz, 2H), 7.37 (d, J=8.0 Hz, 2H), 7.30 (t, J=6.5 Hz, 2H), 2.98 (d, J=4.5 Hz, 3H), 2.32 (brs, 3H); ¹³C NMR (DMSO-d₆, 125 MHz) δ162.5, 157.5, 151.5, 151.3, 149.2, 148.5, 143.9, 138.7, 138.7, 138.1, 135.9, 128.8, 125.4, 124.4, 121.9, 120.3, 114.1, 114.0; MS m/z: 433.21 [M+1]⁺.

### 2-(4-((4-(Tert-butyl)phenyl)sulfonamido)phenyl)-N-methyl-1,5-naphthyridine-4-carboxamide (Compound 1p)

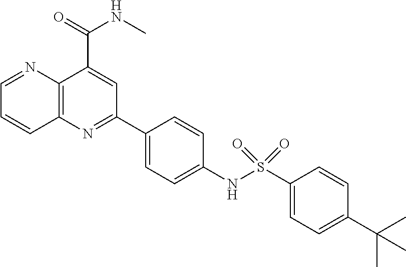 [SMILES: CNC(=O)c1cc(-c2ccc(NS(=O)(=O)c3ccc(C(C)(C)C)cc3)cc2)nc2cccnc12]

Purification done by normal phase column chromatography, mobile phase: ethyl acetate/hexane 10:90 v/v followed by ethyl acetate/hexane 56:44 v/v; Spectral data of the target compounds: Yield: 21%; ¹H NMR (DMSO-d₆, 500 MHz) δ10.69 (brs, 1H), 10.12 (d, J=5.0 Hz, 1H), 9.04 (dd, J=1.5 Hz, 4.0 Hz, 1H), 8.66 (brs, 1H), 8.54 (dd, J=2.0 Hz, 8.5 Hz, 1H), 8.19 (dd, J=2.0 Hz, 7.0 Hz, 2H), 7.89 (dd, J=4.0 Hz, J=8.5 Hz, 1H), 7.79 (dd, J=1.5 Hz, J=6.5 Hz, 2H), 7.60 (dd, J=2.0 Hz, 7.0 Hz, 2H), 7.34-7.32 (m, 2H), 2.98 (d, J=5.0 Hz, 3H), 1.25 (brs, 9H); MS m/z: 475.31 [M+1]⁺.

### 2-(4-((3,5-Bis(trifluoromethyl)phenyl)sulfonamido)phenyl)-N-methyl-1,5-naphthyridine-4-carboxamide (Compound 1q)

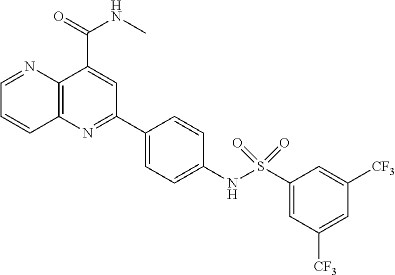 [SMILES: CNC(=O)c1cc(-c2ccc(NS(=O)(=O)c3cc(C(F)(F)F)cc(C(F)(F)F)c3)cc2)nc2cccnc12]

Purification done by normal phase column chromatography, mobile phase: ethyl acetate/hexane 10:90 v/v followed by ethyl acetate/hexane 65:35 v/v; Spectral data of the target compounds: Yield: 15%;¹H NMR (DMSO-d₆, 500 MHz) δ10.30 (brs, 1H), 10.19-10.16 (m, 1H), 9.06 (dd, J=1.5 Hz, J=4.0 Hz, 1H), 8.77 (brs, 1H), 8.59 (dd, J=1.5 Hz, 8.5 Hz, 1H), 8.32-8.29 (m, 1H), 7.99 (brs, 1H), 7.92 (dd, J=4.0 Hz, 8.5 Hz, 1H), 7.80 (d, J=9.0 Hz, 1H), 7.74 (d, J=9.0 Hz, 2H), 7.59 (t, J=8.0 Hz, 1H), 7.49 (d, J=8.0 Hz, 1H), 3.01 (d, J=4.5 Hz, 3H); MS m/z: 554.97 [M+1]⁺.

### General Synthesis of 8a,b

To a solution of compound 6a,b (0.5 mmol) in DCM (3.5 mL), TEA (126 mg, 1.25 mmol) was added at 0° C. The mixture was stirred at the same temperature for 30 min. A solution of phenyl chloroformate (7, 118 mg, 0.75 mmol) in DCM (3.5 mL) was slowly added thereto. The reaction mixture was stirred at room temperature for 1 h. The mixture was then extracted by DCM (3×20 mL) and saturated aqueous NaHCO3 (35 mL). The organic layer was collected, dried over anhydrous Na₂SO₄, and was dried under reduced pressure.

### Phenyl (4-(4-(methylcarbamoyl)-1,5-naphthyridin-2-yl)phenyl)carbamate (Compound 8a)

Purification done by normal phase column chromatography, mobile phase: DCM followed by MeOH/DCM 1.5:98.5 v/v; Spectral data of the target compounds: Yield: 77%; ¹H NMR (DMSO-d₆, 500 MHz) δ10.15 (d, J=5.0 Hz, 1H), 9.04 (dd, J=2.0 Hz, 4.5 Hz, 1H), 8.74 (brs, 1H), 8.59 (dd, J=2.0 Hz, 9.0 Hz, 1H), 8.31 (d, J=9.0 Hz, 2H), 7.90 (d, J=4.0 Hz, J-8.5 Hz, 1H), 7.73 (d, J=9.0 Hz, 2H), 7.47-7.44 (m, 2H), 7.30-7.26 (m, 3H), 3.00 (d, J=4.5 Hz, 3H); ¹³C NMR (DMSO-d₆, 125 MHz) δ 164.6, 157.4, 157.3, 151.4, 149.2, 143.7, 139.0, 137.9, 137.6, 129.7, 129.5, 129.4, 128.6, 128.2, 125.1, 124.5, 122.0, 121.3, 121.0, 118.8, 115.2, 133.8, 26.3; MS m/z: 399.23 [M+1]⁺.

### Phenyl (4-(4-(isopropylcarbamoyl)-1,5-naphthyridin-2-yl)phenyl)carbamate (Compound 8b)

Purification done by normal phase column chromatography, mobile phase: DCM followed by MeOH/DCM 2:98 v/v; Spectral data of the target compounds: Yield: 64%; ¹H NMR (DMSO-d₆, 500 MHz) δ10.37 (brs, 1H), 9.33 (brs, 1H), 8.94 (dd, J=1.5 Hz, 4.0 Hz, 1H), 8.64 (brs, 1H), 8.46 (dd, J=1.0 Hz, 8.5 Hz, 1H), 8.03 (d, J=8.5 Hz, 2H), 7.82 (dd, J=4.0 Hz, 8.5 Hz, 1H), 7.15 (t, J=8.5 Hz, 1H), 6.77-6.70 (m, 4H), 5.75 (m, 2H), 4.20 (dd, J=6.5 Hz, 13.5 Hz, 1H), 1.29 (d, J=6.5 Hz, 6H); ¹³C NMR (DMSO-d₆, 125 MHz) δ163.1, 157.4, 157.3, 151.4, 149.3, 143.8, 139.1, 137.7, 137.7, 129.4, 128.7, 125.1, 124.5, 121.1, 118.9, 115.3, 113.9, 22.5; MS m/z: 427.29 [M+1]⁺.

### General Synthesis of 11a-c

A mixture of compound 9 (660 μL, 0.02 mmol) and the appropriate amine (morpholine, N-methylpiperazine, or N-ethylpiperazine) (10a-c, 0.07 mmol), and TEA (1.4 mL) in THF (2 mL) was stirred at room temperature for 5 min. The reaction mixture was stirred overnight at room temperature. After completion, the organic solvent was removed under reduced pressure and then mixture was extracted by ethyl acetate (3×10 mL) and ice-cooled distilled water (20 mL). The organic layer was collected, dried over anhydrous Na2SO4 and was dried under reduced pressure

### General Synthesis of 12a-c

A mixture of compound 11a-c (0.12 mmol) and Pd/C (0.011 mmol, 5%) in THF (2 mL) was stirred at room temperature for 2 h under H_{2 (g).} Pd/C was filtered out and the solvent was evaporated to obtain the crude product. It was used in the next step without further purification.

### General Synthesis and Spectral Data of 13a-f

A mixture of 12a-c (0.06 mmol), 4-5 molecular sieves, and DIPEA (230 μL) in DMF (200 μL) was stirred at room temperature for 15 min. Compound 8a,b (0.08 mmol) was added and stirred at 40° C. under N_{2 (g)} overnight. The crude product was extracted by ethyl acetate (3×10 mL) and ice-cooled distilled water (20 mL). The organic layer was collected, dried over anhydrous Na₂SO₄, and was dried under reduced pressure.

### N-Methyl-2-(4-(3-(4-morpholino-3-(trifluoromethyl)phenyl)ureido)phenyl)-1,5-naphthyridine-4-carboxamide (Compound 13a)

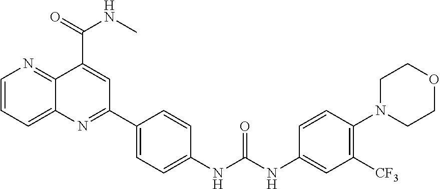 [SMILES: CNC(=O)c1cc(-c2ccc(NC(=O)Nc3ccc(N4CCOCC4)c(C(F)(F)F)c3)cc2)nc2cccnc12]

Purification done by normal phase column chromatography, mobile phase: DCM followed by MeOH/DCM 1.8:98.2 v/v; Spectral data of the target compounds: Yield: 31%; ¹H NMR (DMSO-d₆, 500 MHz) δ10.18 (brs, 1H), 9.11-9.03 (m, 3H), 8.74 (brs, 1H), 8.57 (d, J=8.5 Hz, 1H), 8.27 (d, J=8.0 Hz, 2H), 7.95-7.89 (m, 2H), 7.70-7.54 (m, 4H), 3.70 (brs, 4H), 3.00 (d, J=4.0, 3H), 2.82 (brs, 4H) ;-¹³C NMR (DMSO-d₆, 125 MHz) δ164.5, 156.7, 152.4, 150.3, 145.9, 143.7, 141.8, 139.2, 138.8, 138.0, 137.0, 130.9, 128.2, 126.4-126.2 (J=29.0 Hz), 125.6, 125.3, 125.0, 123.1, 122.8, 121.3, 120.4, 118.4, 116.3, 116.2; 66.7, 53.5, 26.3; MS m/z: 551.37 [M+1]⁺.

### N-Methyl-2-(4-(3-(4-(4-methylpiperazin-1-yl)-3-(trifluoromethyl)phenyl)ureido)phenyl)-1,5-naphthyridine-4-carboxamide (Compound 13b)

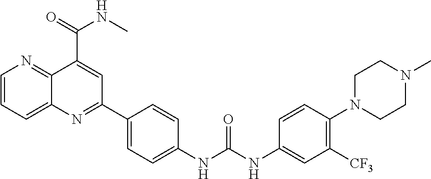 [SMILES: CNC(=O)c1cc(-c2ccc(NC(=O)Nc3ccc(N4CCN(C)CC4)c(C(F)(F)F)c3)cc2)nc2cccnc12]

Purification done by normal phase column chromatography, mobile phase: DCM 100 v/v followed by MeOH/DCM 1.5:98.5 v/v; Spectral data of the target compounds: Yield: 35%; ¹H NMR (Acetone-d₆, 500 MHz) δ10.81 (d, J=4.0 Hz, 1H), 9.02-9.01 (m, 2H), 8.72 (brs, 2H), 8.58 (dd, J=1.5 Hz, 8.5 Hz, 1H), 8.33 (d, J=8.5 Hz, 2H), 8.04 (brs, 1H), 7.88 (dd, J=4.5 Hz, 8.5 Hz, 1H), 7.80 (dd, J=2.0 Hz, 7.0 Hz, 2H), 7.74 (dd, J=2.0 Hz, 8.5 Hz, 1H), 7.50 (d, J=8.5 Hz, 1H), 3.11 (d, J=5.0 Hz, 3H), 2.96 (brs, 4H), 2.64 (brs, 4H), 2.38 (brs, 3H); ¹³C NMR (Acetone-d₆, 125 MHz) δ164.9, 158.3, 153.2, 150.7, 147.9, 145.2, 143.0 140.8, 139.5, 138.0-137.8 (J=26.5 Hz), 132.8, 129.1, 126.2, 126.0, 125.8, 123.9, 123.4, 119.5, 119.4, 117.8, 117.7, 56.3, 54.4, 46.4, 26.6; MS m/z: 564.3 [M+1]⁺.

### 2-(4-(3-(4-(4-Ethylpiperazin-1-yl)-3-(trifluoromethyl)phenyl)ureido)phenyl)-N-methyl-1,5-naphthyridine-4-carboxamide (Compound 13c)

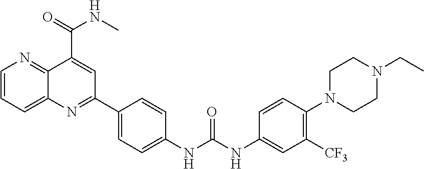 [SMILES: CCN1CCN(c2ccc(NC(=O)Nc3ccc(-c4cc(C(=O)NC)c5ncccc5n4)cc3)cc2C(F)(F)F)CC1]

Purification done by normal phase column chromatography, mobile phase: DCM followed by MeOH/DCM 1.0:99 v/v; Spectral data of the target compounds: Yield: 43%; ¹H NMR (DMSO-d₆, 500 MHz) δ10.18 (d, J=4.5 Hz, 1H), 9.15 (dd, J=4.5 Hz, 19.5, 2H), 9.03 (dd, J=1.5 Hz, 4.0 Hz, 1H), 8.74 (brs, 1H), 8.57 (d, J=8.5 Hz, 1H), 8.27 (d, J=9.0 Hz, 2H), 7.95 (d, J=2.0 Hz, 1H), 7.89 (dd, J=4.5 Hz, 8.5 Hz, 1H), 7.69 (d, J=8.5 Hz, 2H), 7.61 (d, J=8.0, 1H), 7.52 (d, J=9.0 Hz, 1H), 3.00 (d, J=5.0 Hz, 5H), 2.85 (brs, 4H), 1.15 (t, J=7.0, 2H), 1.04 (t, J=7.0 Hz, 3H) ; ¹³C NMR (DMSO-d₆, 125 MHz) δ176.2, 164.6, 156.7, 152.4, 143.7, 141.8, 140.1, 139.2-138.8 (J=54.0 Hz), 138.0, 131.0, 128.2, 125.4, 123.1, 121.4, 118.4, 90.9, 77.4, 52.3, 51.4, 26.4; MS m/z: 578.15 [M+1]⁺.

### N-Isopropyl-2-(4-(3-(4-morpholino-3-(trifluoromethyl)phenyl)ureido)phenyl)-1,5-naphthyridine-4-carboxamide (Compound 13d)

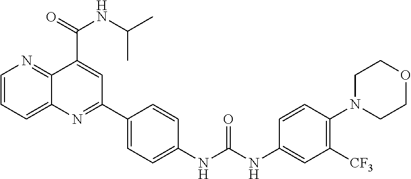 [SMILES: CC(C)NC(=O)c1cc(-c2ccc(NC(=O)Nc3ccc(N4CCOCC4)c(C(F)(F)F)c3)cc2)nc2cccnc12]

Purification done by normal phase column chromatography, mobile phase: DCM followed by MeOH/DCM 1.2:98.8 v/v; Spectral data of the target compounds: Yield: 51%; ¹H NMR (Acetone-d₆, 500 MHz) δ10.87 (d, J=7.0 Hz, 1H), 9.05-9.03 (m, 2H), 8.59 (dd, J=1.5 Hz, 8.5 Hz, 1H), 8.50 (d, J=18.0 Hz, 2H), 8.36-8.33 (m, 2H), 8.00 (d, J=2.5 Hz, 1H), 7.89 (dd, J=4.0 Hz, 8.5 Hz, 1H), 7.81-7.76 (m, 3H), 7.53 (d, J=8.5 Hz, 1H), 4.32 (d, J=7.5 Hz, 1H), 3.75 (t, J=4.5 Hz, 4H), 2.88 (t, J=4.5 Hz, 4H), 1.36 (d, J=6.5 Hz, 6H); ¹³C NMR (Acetone-d₆, 125 MHz) δ163.4, 158.3, 150.6, 147.5, 145.3, 139.6, 138.3-138.0 (J=40.0 Hz), 133.4, 132.8, 132.1, 151.1, 129.1, 126.2, 125.7, 124.0, 123.4, 119.5, 117.7, 107.9, 67.9, 54.8, 42.6, 22.9; MS m/z: 579.25 [M+1]⁺.

### N-Isopropyl-2-(4-(3-(4-(4-methylpiperazin-1-yl)-3-

(trifluoromethyl)phenyl)ureido)phenyl)-1,5-naphthyridine-4-carboxamide (Compound 13e)

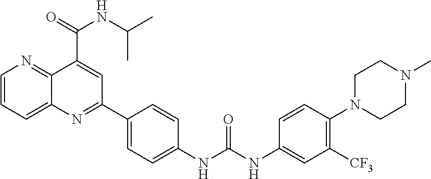 [SMILES: CC(C)NC(=O)c1cc(-c2ccc(NC(=O)Nc3ccc(N4CCN(C)CC4)c(C(F)(F)F)c3)cc2)nc2cccnc12]

Purification done by normal phase column chromatography, mobile phase: DCM followed by MeOH/DCM 0.9:99.1 v/v; Spectral data of the target compounds: Yield: 65%; ¹H NMR (CDCl₃-MeOD-d₄, 500 MHz) δ11.15 (d, J=9.5 Hz, 1H), 8.95 (brs, 1H), 8.85 (dd, J=2.0 Hz, 5.5 Hz, 1H), 8.49 (dd, J=2.0 Hz, 10.5 Hz, 1H), 8.14 (d, J=11.0 Hz, 2H), 7.66 (dd, J=5.0 Hz, 10.5 Hz, 2H), 7.59-7.56 (m, 3H), 7.31-7.26 (m, 1H), 4.32 (dd, J=8.5 Hz, 15.0 Hz, 1H), 2.90 (brs, 4H), 2.61 (d, J=3.5 Hz, 4H), 2.35 (brs, 3H), 1.33 (d, J=8.0 Hz, 6H); ¹³C NMR (CDCl₃-MeOD-d₄, 125 MHz) δ163.7, 163.6, 158.3, 153.0, 153.0, 149.2, 146.5, 144.3, 141.5, 141.4, 140.2, 138.6, 136.5, 136.1, 132.2, 128.6, 128.1-127.9 (J=29.0 Hz), 125.0, 124.9, 124.4, 123.8, 123.3, 123.1, 122.7, 119.0, 118.9, 117.6, 117.5, 55.3, 52.7, 45.6, 42.3 , 42.2, 22.7, 22.6; MS m/z: 592.29 [M+1]⁺.

### 2-(4-(3-(4-(4-Ethylpiperazin-1-yl)-3-(trifluoromethyl)phenyl)ureido)phenyl)-N-isopropyl-1,5-naphthyridine-4-carboxamide (Compound 13f)

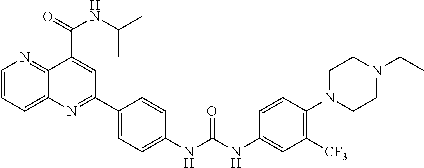 [SMILES: CCN1CCN(c2ccc(NC(=O)Nc3ccc(-c4cc(C(=O)NC(C)C)c5ncccc5n4)cc3)cc2C(F)(F)F)CC1]

Purification done by normal phase column chromatography, mobile phase: DCM followed by MeOH/DCM 0.6:99.4 v/v; Spectral data of the target compounds: Yield: 19%; ¹H NMR (CDCl₃-MeOD-d₄, 500 MHz) δ11.17 (d, J=9.0 Hz, 1H), 8.94 (d, J=3.5 Hz, 1H), 8.85 (t, J=2.0 Hz, 1H), 8.48 (d, J=10.5 Hz, 1H), 8.14-8.12 (m, 2H), 7.67-7.64 (m, 2H), 7.56 (dd, J=8.5 Hz, 10.5 Hz, 3H), 7.31 (d, J=10.5 Hz, 1H), 4.31 (d, J=8.0 Hz, 1H), 2.90 (brs, 4H), 2.62-2.50 (m, 6H), 1.33-1.32 (m, 6H), 1.12-1.01 (m, 3H); ¹³C NMR (CDCl₃-MeOD-d₄, 125 MHZ) 8163.7, 163.6, 158.3, 153.0, 152.9, 149.2, 146.5, 144.3, 141.5, 141.4, 140.1, 138.6, 136.5, 136.4, 136.1, 132.1, 128.6, 128.3, 128.1, 127.8, 127.6-127.0 (J=74.0 Hz), 125.0, 124.9, 124.4, 123.8, 123.2, 123.1, 122.7, 120.5, 118.9, 118.8, 117.5, 117.5, 53.0, 52.7, 52.4, 42.2, 22.6; MS m/z: 606.47 [M+1]⁺.

### Synthesis of 1-(4-nitro-2-(trifluoromethyl)phenyl)piperazine (Compound 15)

To compound 14 (246 mg, 2.9 mmol) dissolved in DMF (5 mL) was added K₂CO₃ (825 mg, 5.98 mmol) followed by compound 9 (500 mg ,2.4 mmol) and stirred at room temperatures for 12 h. After completion, the reaction mixture was quenched in ice and extracted by ethyl acetate (3 × 10 mL) and ice-cooled distilled water (20 mL). The organic layer was collected, dried over anhydrous Na₂SO₄ and was dried under reduced pressure.

Purification done by normal phase column chromatography, mobile phase:ethyl acetate/hexane 10:90 v/v followed by ethyl acetate/hexane 30:70 v/v; Spectral data of the target compounds: Yield: 71%; ¹H NMR (CDCl₃, 500 MHz) δ8.50 (d, J=3.0 Hz, 1H), 8.32 (dd, J=2.5 Hz, 9.0 Hz, 1H), 7.27 (d, J=10.0 Hz, 1H), 3.11 (dd, J=4.5 Hz, 7.0 Hz, 4H), 3.04 (dd, J=2.5 Hz, 5.0 Hz, 4H); C NMR (CDCl₃, 125 MHz) δ157.4, 142.2, 128.0, 126.5, 124.9, 124.8, 124.8, 124.7, 124.6, 124.4, 124.4, 124.2, 122.5, 122.2, 120.0, 54.1, 46.1; MS m/z: 276.23 [M+1]⁺.

### Synthesis of tert-butyl 4-(4-nitro-2-(trifluoromethyl)phenyl)piperazine-1-carboxylate (Compound 16)

A mixture of compound 15 (440 mg, 1.6 mmol), Boc anhydride (349.2 mg, 1.6 mmol), and TEA (111 μL) in DCM (4 mL) was stirred at room temperature for 4 h. The crude compound was extracted with DCM (3×15 mL) and ice-cooled distilled water (15 mL). The organic layer was collected, dried over anhydrous Na₂SO₄ and was dried under reduced pressure. Purification was done by normal phase column chromatography, mobile phase:ethyl acetate/hexane 10:90 v/v followed by ethyl acetate/hexane 35:65 v/v; Spectral data of the target compounds: Yield: 64%; H NMR (CDCl₃, 500 MHz) δ8.53 (d, J=2.5 Hz, 1H), 8.35 (dd, J=2.5 Hz, 8.5 Hz, 1H), 7.31-7.26 (m, 1H), 3.60 (t, J=5.0 Hz, 4H), 3.06 (t, J=5.0 Hz, 4H), 1.56-1.49 (m, 9H).

### Synthesis of tert-butyl 4-(4-amino-2-(trifluoromethyl)phenyl)piperazine-1-carboxylate (Compound 17)

A mixture of compound 16 (150 mg, 0.5 mmol) and Pd/C (0.011 mmol, 5%) in THF (3 mL) and stirred at room temperature for 2 h under H_{2 (g)}. Pd/C was filtered out, and the organic solvent was evaporated to collect the crude product. Purification was done by normal phase column chromatography, mobile phase:ethyl acetate/hexane 10:90 v/v followed by ethyl acetate/hexane 50:50 v/v; Spectral data of the target compounds: Yield: 47%; ¹H NMR (CDCl₃, 500 MHz) δ7.26 (brs, 2H), 7.13 (d, J=8.5 Hz, 1H), 6.90 (d, J=3.0 Hz, 1H), 6.79 (dd, J=2.5 Hz, J=8.5 Hz, 1H), 3.51 (brs, 4H), 2.76 (d, J=4.0 Hz, 4H), 1.47 (brs, 9H).

### Synthesis of 13g and 13j

A mixture of compound 17 (11 mg, 0.03 mmol), 4-5 molecular sieves, and DIPEA (230 μL) in DMF (200 μL) was stirred at room temperature for 15 min. Compound 8a,b (21.3 mg, 0.05 mmol) was added, and the mixture was stirred for 40° C. under N_{2 (g)}overnight. The crude compound was extracted by ethyl acetate (3×10 mL) and ice-cooled distilled water (20 mL). The organic layer was collected, dried over anhydrous Na₂SO₄ and was dried under reduced pressure.

### Tert-butyl4-(4-(3-(4-(4-(methylcarbamoyl)-1,5-naphthyridin-2-yl)phenyl)ureido)-2(trifluoromethyl)phenyl)piperazine-1-carboxylate (Compound 13g)

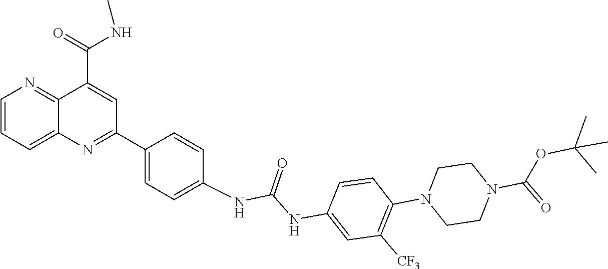 [SMILES: CNC(=O)c1cc(-c2ccc(NC(=O)Nc3ccc(N4CCN(C(=O)OC(C)(C)C)CC4)c(C(F)(F)F)c3)cc2)nc2cccnc12]

Purification done by normal phase column chromatography, mobile phase: DCM/MeOH 0:100 v/v followed by DCM/MeOH 1.5 :98.5 v/v; Spectral data of the target compounds: Yield: 71%; ¹H NMR (CDCl₃, 500 MHz) δ 11.50 (d, J=5.0 Hz, 1H), , 8.94 (brs, 1H), 8.92 (dd, J=1.5 Hz, 4.0 Hz, 1H), 8.56 (dd, J=1.5 Hz, 8.5 Hz, 1H), 8.37 (brs, 1H), 8.30 (brs, 1H), 8.06 (d, J=9.0 Hz, 2H), 7.79 (dd, J=2.5 Hz, 8.5 Hz,1H), 7.73 (dd, J=4.5 Hz, 8.5 Hz, 1H), 7.65 (d, J=8.5 Hz, 2H), 7.61 (d, J=2.5 Hz, 1H), 7.27 (t, J=8.5 Hz, 1H), 3.54 (brs, 4H), 3.26 (brs, J=4.5 Hz, 3H), 2.82 (brs, 4H), 1.49 (brs, 9H); 13C NMR (CDCl₃, 125 MHz) δ166.1, 158.3, 155.0, 153.0, 149.2, 147.0, 144.5, 141.3, 139.8, 139.1, 136.2, 135.3, 132.4, 128.7, 128.1-127.6 (J=114.5 Hz), 127.0, 124.9, 124.8, 124.6, 123.9, 123.3, 122.6, 120.4, 119.5, 118.3, 118.2, 79.8, 53.4, 29.7, 28.5, 27.1; MS m/z: 650.36 [M+1]⁺

### Tert-butyl 4-(4-(3-(4-(4-(isopropylcarbamoyl)-1,5-naphthyridin-2-yl)phenyl)ureido)-2-(trifluoromethyl) phenyl) piperazine-1-carboxylate (Compound 13j)

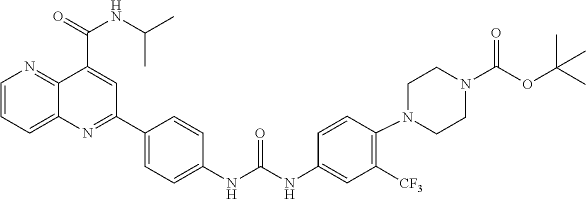 [SMILES: CC(C)NC(=O)c1cc(-c2ccc(NC(=O)Nc3ccc(N4CCN(C(=O)OC(C)(C)C)CC4)c(C(F)(F)F)c3)cc2)nc2cccnc12]

Purification done by normal phase column chromatography, mobile phase: DCM followed by MeOH/DCM 1.5:98.5 Spectral data of the target compounds: Yield: v/v; 51%; ¹H NMR (Acetone-d₆, 500 MHz) δ10.87 (d, J=7.0 Hz, 1H), 9.05-9.02 (m, 2H), 8.59 (dd, J=1.5 Hz, 8.5 Hz, 1H), 8.49 (d, J=17.0 Hz, 2H), 8.33 (d, J=8.5, 2H), 8.00 (d, J=2.0 Hz, 1H), 7.89 (dd, J=4.0 Hz, 8.5 Hz, 1H), 7.80-7.75 (m, 3H), 7.50 (d, J=9.0 Hz, 1H), 4.32 (d, J=6.5 Hz, 13.5 Hz, 1H), 3.52 (brs, 4H), 2.86-2.79 (m, 4H), 1.47 (brs, 9H), 1.36 (d, J=6.5 Hz, 6H); ¹³C NMR (Acetone-d₆, 125 MHz) δ163.3, 158.3, 155.1, 153.1, 153.1, 150.6, 147.4, 145.2, 142.9, 142.8, 140.9, 139.6, 138.3, 138.2, 137.9, 132.8, 129.1, 128.1, 126.2-126.1 (J=12.5 Hz), 125.7, 123.9, 123.8, 123.4, 119.5, 119.4, 117.8, 117.7, 117.7, 117.6, 54.3, 42.6, 28.6, 22.9; MS m/z: 678.49 [M+1]⁺.

### Synthesis of 13h and 13k

A mixture of compound 13g/13j (0.03 mmol) and trifluoroacetic acid (40 μL) in DCM (200 μL) was stirred at room temperature overnight. The mixture was then extracted by DCM (3×20 mL) and saturated aqueous NaHCO₃ (35 mL). The organic layer was collected, dried over anhydrous Na₂SO₄, and was dried under reduced pressure to obtain the crude product.

### N-Methyl-2-(4-(3-(4-(piperazin-1-yl)-3-(trifluoromethyl)phenyl)ureido)phenyl)-1,5-naphthyridine-4-carboxamide (Compound 13h)

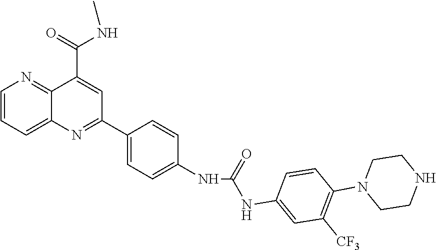 [SMILES: CNC(=O)c1cc(-c2ccc(NC(=O)Nc3ccc(N4CCNCC4)c(C(F)(F)F)c3)cc2)nc2cccnc12]

Purification done by normal phase column chromatography, mobile phase: DCM/MeOH 0:100 v/v followed by DCM/MeOH 3:97 v/v; Spectral data of the target compounds: Yield: 63%; ¹H NMR (Acetone-d₆, 500 MHz) δ10.81 (brs, 1H), 9.01 (d, J=5.0 Hz, 2H), 8.56 (d, J=8.0 Hz, 1H), 8.29-8.23 (m, 3H), 7.94-7.85 (m, 4H), 7.50 (d, J=8.0 Hz, 1H), 3.54 (brs, 4H), 3.29 (d, J=14.0 Hz, 4H), 3.10 (d, J=4.0 Hz, 3H); ¹³C NMR (Acetone-d₆, 125 MHz) δ165.0, 158.5, 153.8, 150.5, 145.2, 145.1, 143.9, 140.7, 140.0, 139.5, 137.6, 132.1, 130.6, 128.9, 128.2-127.8 (J=28.5 Hz), 126.1, 126.0, 125.8, 125.7, 124.0, 123.6, 123.4, 119.3, 117.3, 117.3, 51.1, 41.3, 26.4, MS m/z: 550.24 [M+1]⁺

### N-Isopropyl-2-(4-(3-(4-(piperazin-1-yl)-3-(trifluoromethyl) phenyl) ureido) phenyl)-1,5-naphthyridine-4-carboxamide (Compound 13k)

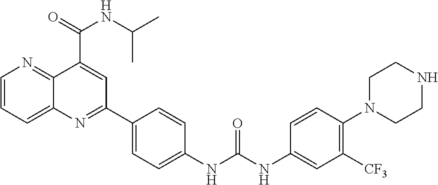 [SMILES: CC(C)NC(=O)c1cc(-c2ccc(NC(=O)Nc3ccc(N4CCNCC4)c(C(F)(F)F)c3)cc2)nc2cccnc12]

Purification done by normal phase column chromatography, mobile phase: DCM followed by MeOH/DCM 1.5:98.5 v/v; Spectral data of the target compounds: Yield: 66%; ¹H NMR (Acetone-d₆, 500 MHz) δ10.87 (d, J=7.0 Hz, 1H), 9.04-9.02 (m, 2H), 8.95 (brs, 1H), 8.58 (dd, J=2.0 Hz, J=8.5 Hz, 1H), 8.32 (d, J=8.5 Hz, 2H), 8.04 (d, J=2.0 Hz, 1H), 7.89 (d, J=4.0 Hz, 1H), 7.87 (d, J=4.0 Hz, 2H), 7.83-7.76 (m, 1H), 7.46 (d, J=8.5 Hz, 1H), 4.35-4.29 (m, 1H), 3.00 (d, J=3.0 Hz, 4H), 2.09-2.04 (m, 4H), 1.37 (brs, 6H); ¹³C NMR (Acetone-d₆, 125 MHz) δ163.3, 158.4, 153.3, 150.6, 148.0, 145.2, 143.2, 140.9, 139.5, 138.2-137.9 (J=45.0 Hz), 132.5, 130.6, 129.0, 128.0, 126.2, 126.0, 125.7, 123.8, 123.4, 119.4, 117.6, 117.5, 55.4, 54.5, 54.3, 47.1, 42.5, 27.8, 22.9; MS m/z: 578.30 [M+1]⁺.

### Synthesis of 13i and 13l

To a solution of compound 13h/13k (0.03 mmol) in DMF (0.5 mL), DIPEA (1.2 μL) was added followed by acyloyl chloride (4 μL). The reaction mixture was stirred at room temperature overnight. The reaction mixture was partitioned between ethyl acetate (3×5 mL) and saline (5 mL). The organic layer was collected, dried over anhydrous Na₂SO₄, and the organic solvent was evaporated under reduced pressure to obtain the crude product.

### 2-(4-(3-(4-(4-Acryloylpiperazin-1-yl)-3-(trifluoromethyl)phenyl)ureido)phenyl)-N-methyl-1,5-naphthyridine-4-carboxamide (Compound 13i)

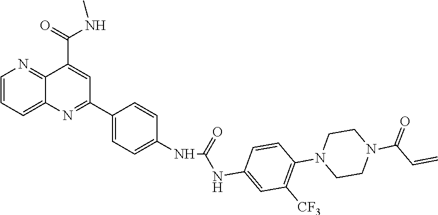 [SMILES: C=CC(=O)N1CCN(c2ccc(NC(=O)Nc3ccc(-c4cc(C(=O)NC)c5ncccc5n4)cc3)cc2C(F)(F)F)CC1]

Purification done by normal phase column chromatography, mobile phase: DCM/MeOH 0:100 v/v followed by DCM/MeOH 2:98 v/v; Spectral data of the target compounds: Yield: 54%; ¹H NMR (CD₂Cl₂, 500 MHz) δ11.17 (d, J=4.5 Hz, 1H), 8.99 (brs, 1H), 8.91 (dd, J=1.5 Hz, 4.5 Hz, 1H), 8.54 (dd, J=1.5 Hz, 8.5 Hz, 1H), 8.22 (d, J=8.5 Hz, 2H), 7.74-7.63 (m, 5H), 7.30 (d, J=8.5 Hz, 1H), 6.63 (dd, J=11.0 Hz, 17.0 Hz, 1H), 6.23 (dd, J=2.0 Hz, 17.0 Hz, 1H), 5.72 (dd, J=2.0 Hz, 10.5 Hz, 1H), 3.77-3.65 (m, 4H), 3.14 (d, J=4.5 Hz, 3H), 2.88 (t, J=4.5 Hz, 4H); ¹³C NMR (CD₂Cl₂, 125 MHz) δ166.3, 165.6, 158.4, 153.1, 149.8, 146.3, 144.7, 141.9, 140.4, 139.0, 137.3, 136.4, 132.5, 128.1-128.0 (J=4.5 Hz), 125.4, 125.0, 123.7, 123.4, 119.1, 117.7, 117.6, 46.9, 43.0, 26.7; MS m/z: 604.18 [M+1]⁺.

### 2-(4-(3-(4-(4-Acryloylpiperazin-1-yl)-3-(trifluoromethyl)phenyl)ureido)phenyl)-N-isopropyl-1,5-naphthyridine-4-carboxamide (Compound 13l)

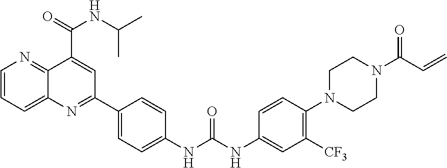 [SMILES: C=CC(=O)N1CCN(c2ccc(NC(=O)Nc3ccc(-c4cc(C(=O)NC(C)C)c5ncccc5n4)cc3)cc2C(F)(F)F)CC1]

Purification done by normal phase column chromatography, mobile phase: DCM followed by MeOH/DCM 2:98 v/v; Spectral data of the target compounds: Yield: 54%; ¹H NMR (Acetone-d₆, 500 MHz) δ10.87 (d, J=7.0 Hz, 1H), 9.04-9.01 (m, 2H), 8.86 (d, J=20.5 Hz, 2H), 8.59-8.56 (m, 1H), 8.33-8.31 (m, 2H), 8.04 (d, J=4.0 Hz, 1H), 7.89-7.86 (m, 1H), 7.81-7.76 (m, 3H), 7.50 (dd, J=5.5 Hz, 7.5 Hz, 1H), 6.81 (dd, J=10.5 Hz, J=16.5 Hz, 1H), 6.21 (dd, J=2.5 Hz, 16.5 Hz, 1H), 5.67 (dd, J=2.5 Hz, 10.5 Hz, 1H), 4.32 (dd, J=7.0 Hz, 14.0 Hz, 1H), 3.75 (brs, 4H), 2.87 (brs , 4H), 1.36 (d, J=6.5 Hz, 6H) ; ¹³C NMR (Acetone-d₆, 125 MHz) δ165.4, 163.4, 158.3, 153.3, 150.6, 146.9, 145.2, 143.1, 140.9, at 139.5, 138.6, 137.8, 132.7, 130.6, 129.1, 128.4-128.1 (J=25.5 Hz), 127.4, 126.3, 126.1, 125.7, 123.9, 123.8, 123.4, 119.4, 117 6, 117.5, 55.2, 54.8, 54.1, 46.9, 43.3, 43.1, 42.6, 40.9, 36.1, 32.6, 27.8 26.3, 23.3, 22.9, 14.4, 13.0; MS m/z: 632.28 [M+1]⁺.

### Synthesis of Compounds 1a-q

The synthetic pathways are illustrated in Schemes 1-3, and the structures of the synthesized derivatives are shown in Tables 1-3.

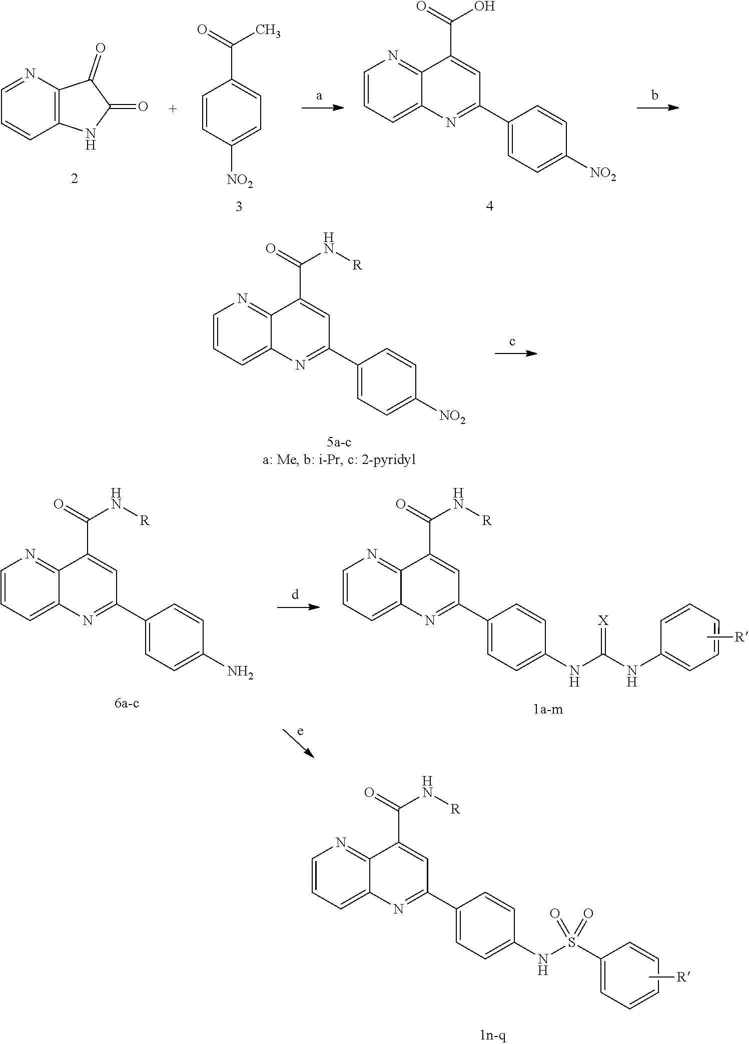

The synthetic pathways are illustrated in Schemes 1-3, and the structures of the synthesized derivatives are shown in Tables 1-3.

### Synthesis of Compounds 13a-f

Reagents and conditions: a) KOH, EtOH, rt, 12 h; b) RNH₂, TBTU, TEA, DMF, —15° C. to rt, 2 h; c) Fe, NH₄Cl, MeOH/H₂O, 80° C., 24 h; d) appropriate aryl isocyanate or isothiocyanate, DMF, rt, overnight; e) appropriate arylsulfonyl chloride, pyridine 0° C. to rt, overnight.

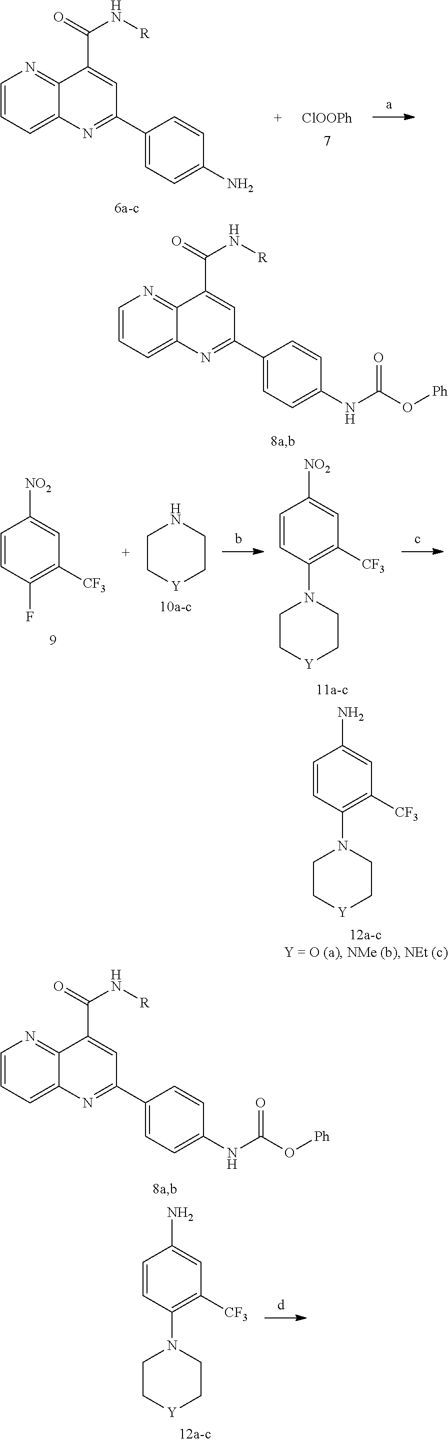

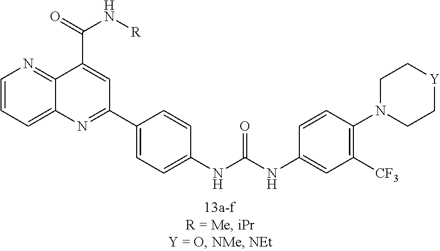

### Synthesis of compounds 13g-13i

Reagents and conditions: a) TEA, DCM, 0° C. to rt, 1h; b) TEA, THF, rt, overnight; c) hydrogen gas, Pd/C, rt, 2 h; d) DIPEA, DMF, 40° C., overnight.

### Materials and Methods

### Testing the Target Compounds Against NCI-60 Panel

It was carried out at the NCI, Bethesda, MD, USA, utilizing their standard protocol.

### Screening Against HCC Cell Lines

### Cell Culture

Two HCC cell lines (Hep3B and HepG2) were used for screening the antiproliferative activity of the synthesized compounds. The cell lines were obtained from the European Collection of Cell Cultures (ECACC, UK) and maintained in Dulbecco's Modified Eagle Medium (DMEM: Sigma-Aldrich) supplemented with 10% fetal bovine serum (FBS) and 1% penicillin/streptomycin and incubated at 37° C. in a 5% CO₂ air atmosphere. In addition, WI-38 normal cell line was used to investigate the safety of these compounds.

### 2-(4,5-Dimethylthiazol-2-yl)-2,5-diphenyl tetrazolium bromide (MTT) Assay

MTT assay was conducted to calculate the IC50 of the compounds following the reported procedure . Briefly, the cells were seeded in 96-well tissue culture plates with a density of 5×104 cells/well and incubated overnight at 37° C. in a 5% CO2 air atmosphere. After that, cells were treated with the newly synthesized compounds or sorafenib as a positive control. DMSO was used as a negative control. After 48 h, the media were aspirated, and replaced with media containing 0.5 mg/mL of MTT tetrazolium dye (Sigma-Aldrich) for 2 h at 37° C. Finally, the media were removed and 200 μL of DMSO was added to solubilize the formazan crystals. Absorbance was then measured at 570 nm using Varioskan Flash multimode plate reader (Thermo Scientific, Massachusetts, USA).

### REFERENCES

[1] H. Frankish, Lancet 361 (2003) 1278.
[2] Belpomme, P. Irigaray, A.J. Sasco, J.A. Newby, V. Howard, R. Clapp, L. Hardell, Int. J. Oncol. 30 (2007) 1037-1049.].
[3] Nair, A.B., Jacob S. (2016). A simple practical guide for dose conversion between animal and human. J Basic Clin Pharma 2016, 7:27-31.

What is claimed is:

1. A compound according to formula I, or pharmaceutically acceptable salt thereof: 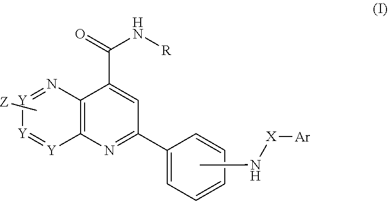
wherein:
R is independently selected from the group consisting of: Me, i-Pr, and 2-pyridil;
X is independently selected from the group consisting of: CO—NH, CS—NH, CO, and SO₂;
Y is independently selected from the group consisting of: CH and CN;
Ar is independently selected from the group consisting of: substituted aromatic, unsubstitued aromatic and heteroaromatic rings;
Z is independently selected from the group consisting of: Alkyl, substituted alkyl, aryl substituted aryl, ether, amin, substituted amine, alogen, and sulfonyl amide.

2. A compound according to formula I, or pharmaceutically acceptable salt thereof, wherein the compound is of formula II: 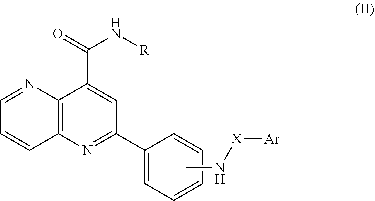
wherein:
R is independently selected from the group consisting of: Me, i-Pr, and 2-pyridil;
X is independently selected from the group consisting of: CO—NH, CS—NH;
Ar is independently selected from the group consisting of: o-Fluoro(trifluoromethyl)benzene, m-Bis(trifluoromethyl)benzene, 4-[o-(Trifluoromethyl)phenyl]morpholine, 4-Methyl-1-[o-(trifluoromethyl)phenyl]piperazine, and 4-Ethyl-1-[o-(trifluoromethyl)phenyl]piperazine.

3. The compound of claim 2, or pharmaceutically acceptable salt thereof, wherein the compound is of formula 1j:

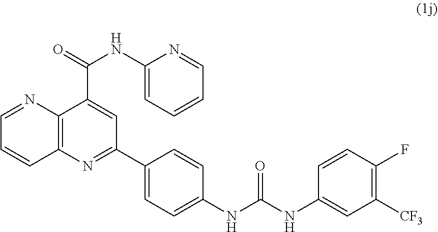 [SMILES: O=C(Nc1ccc(-c2cc(C(=O)Nc3ccccn3)c3ncccc3n2)cc1)Nc1ccc(F)c(C(F)(F)F)c1]

4. The compound of claim 2, or pharmaceutically acceptable salt thereof, wherein the compound is of formula 1m:

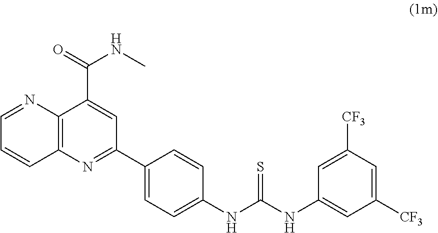 [SMILES: CNC(=O)c1cc(-c2ccc(NC(=S)Nc3cc(C(F)(F)F)cc(C(F)(F)F)c3)cc2)nc2cccnc12]

5. The compound of claim 2, or pharmaceutically acceptable salt thereof, wherein the compound is of formula 13a:

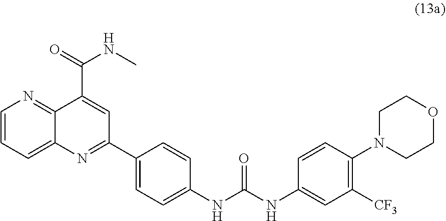 [SMILES: CNC(=O)c1cc(-c2ccc(NC(=O)Nc3ccc(N4CCOCC4)c(C(F)(F)F)c3)cc2)nc2cccnc12]

6. The compound of claim 2, or pharmaceutically acceptable salt thereof, wherein the compound is of Formula 13b:

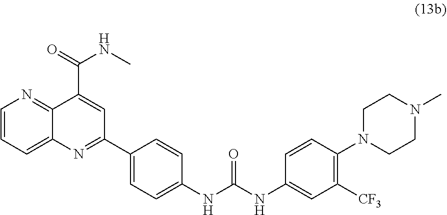 [SMILES: CNC(=O)c1cc(-c2ccc(NC(=O)Nc3ccc(N4CCN(C)CC4)c(C(F)(F)F)c3)cc2)nc2cccnc12]

7. The compound of claim 2, or pharmaceutically acceptable salt thereof, wherein the compound is of Formula 13c:

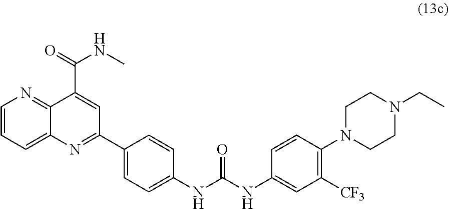 [SMILES: CCN1CCN(c2ccc(NC(=O)Nc3ccc(-c4cc(C(=O)NC)c5ncccc5n4)cc3)cc2C(F)(F)F)CC1]

8. The compound of claim 2, or pharmaceutically acceptable salt thereof, wherein the compound is of Formula 13e:

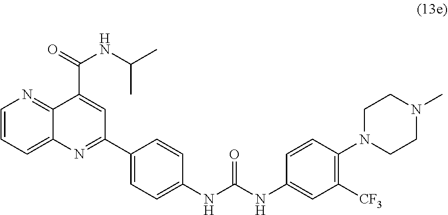 [SMILES: CC(C)NC(=O)c1cc(-c2ccc(NC(=O)Nc3ccc(N4CCN(C)CC4)c(C(F)(F)F)c3)cc2)nc2cccnc12]

9. The compound of claim 2, or pharmaceutically acceptable salt thereof, wherein the compound is of Formula 13f:

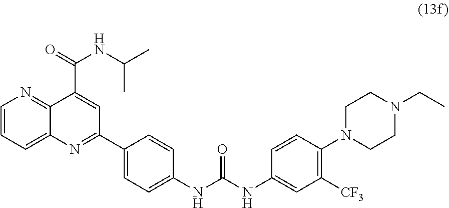 [SMILES: CCN1CCN(c2ccc(NC(=O)Nc3ccc(-c4cc(C(=O)NC(C)C)c5ncccc5n4)cc3)cc2C(F)(F)F)CC1]

10. A pharmaceutical composition, comprising a therapeutically effective amount of one or more of the compounds, or pharmaceutically acceptable salts thereof, of claims 1-9, and one or more pharmaceutical excipients.

11. A method of treating a subject afflicted by a disease associated with an altered expression of a kinase, comprising administering to the subject in need thereof a therapeutically effective amount of the compound, a pharmaceutically acceptable salt thereof, of claims 1-9, and one or more pharmaceutical excipients.

12. The method of claim 11, wherein the subject is afflicted by a cancer associated with an abnormal expression of a kinase selected from the group consisting of protein tyrosine kinase and FMS kinase.

13. The method of claim 11, wherein the subject is afflicted by an inflammatory disease associated with an abnormal expression of a kinase selected from the group consisting of protein tyrosine kinase and FMS kinase.

14. The method of claims 11, wherein the subject is afflicted by a disease associated with an abnormal expression of a kinase, independently selected from the group consisting of cancer, arthritis, Alzheimer, and inflammation, diabetes, psoriasis, angiogenesis, restenosis, and rheumatoid arthritis.

15. The method of claims 11-14, wherein the subject is a mammal.

16. The method of claims 15, wherein the mammal is a human.

17. The method of claim 11-14, comprising inhibiting a kinase with an altered expression by contacting the kinase with an effective amount of the compound of claims 1-9.

18. A kit for treating a subject with a cancer associated with an altered expression of one or more kinases, comprising the compound of claims 1-9, or a pharmaceutically acceptable salt thereof;
one or more pharmaceutical excipients.

19. A method of synthesizing one or more of compounds 1j, 1m, 13a, 13b, 13c, 13e, and 13f of claims 3-9, comprising:
a. forming a reaction mixture from ingredients comprising Azaisatin 2, K₂CO₃, compound 3, and KOH, to produce compound 4; forming a reaction mixture from ingredients comprising compound 4, TEA, TBTU, and R in the amine is independently selected from the group consisting of: alkyl, substituted alkyl, aryl, substituted aryl, substituted heteroaromatic rings, unsubstituted heteroaromatic rings to obtain one or more of compounds 5a, 5b, and 5c;
b. forming a reaction mixture from ingredientes comprising one or more of compounds 5a, 5b, and 5c, NH₄Cl and Fe powder to formo one or more of compounds 6a, 6b, and 6c;
c. forming a reaction mixture from ingredients comprising one or more of compounds 6a, 6b, 6c, and a compound selected from the group consisting of isocyanate and isothiocyanate;
d. reacting one or more of compounds 6a and 6b and phenyl chloroformate;
e. reacting one or more of compounds 9, 10a, 10b, and 10c, with an amine selected from the group consisting of morpholine, N-methylpiperazine, and N-ethylpiperazine, and TEA to form one or more of compounds 11a, 11b, and 11c;
f. reacting one or more of compounds 11a, 11b, and 11c with Pd/C, to form one or more of compounds 12a, 12b, and 12c;
g. reacting one or more of compounds 12a, 12b, and 12c with DIPEA to form one or more of compounds 13a, 13b, 13c, 13e, and 13f.
